SEQUENCE LISTING (11 sequences)

SEQ ID NO: 1 - protein, 433 aa, Corynebacterium glutamicum
MTVPTLNKASEKIASRETLRLCTAGSVDDGKSTFVGRLLHDTKSVLADQLASVERTSADRGFEGLDLSLLVDGLRAEREQGITIDVAYRYFATDKRTFILADTPGHVQYTRNTVTGVSTSQVVVLLVDARHGVVEQTRRHLSVSALLGVRTVILAVNKIDLVDYSEEVFRNIEKEFVSLASALDVTDTHVVPISALKGDNVAEPSTHMDWYAGPTVLEILENVEVSRGRAHDLGFRFPIQYVIREHATDYRGYAGTINAGSISVGDTVHLPEGRTTQVTHIDSADGSLQTASVGEAVVLRLAQEIDLIRGELIAGSDRPESVRSFNATVVGLADRTIKPGAAVKVRYGTELVRGRVAAIERVLDIDGVNDNEAPETYGLNDIAHVRIDVAGELEVEDYAARGAIGSFLLIDQSSGDTLAAGLVGHRLRNNWSI

SEQ ID NO: 2 - protein, 261 aa, Corynebacterium glutamicum
MSFQLVNALKNTGSVKDPEISPEGPRTTTPLSPEVAKHNEELVEKHAAALYDASAQEILEWTAEHTPGAIAVTLSMENTVLAELAARHLPEADFLFLDTGYHFKETLEVARQVDERYSQKLVTALPILKRTEQDSIYGLNLYRSNPAACCRMRKVEPLAASLSPYAGWITGLRRADGPTRAQAPALSLDATGRLKISPIITWSLEETNEFIADNNLIDHPLTHQGYPSIGCETCTLPVAEGQDPRAGRWAGNAKTECGLHS

SEQ ID NO: 3 - protein, 561 aa, Corynebacterium glutamicum
MTTTTGSARPARAARKPKPEGQWKIDGTEPLNHAEEIKQEEPAFAVKQRVIDIYSKQGFSSIAPDDIAPRFKWLGIYTQRKQDLGGELTGQLPDDELQDEYFMMRVRFDGGLASPERLRAVGEISRDYARSTADFTDRQNIQLHWIRIEDVPAIWEKLETVGLSTMLGCGDVPRVILGSPVSGVAAEELIDATPAIDAIRERYLDKEEFHNLPRKFKTAITGNQRQDVTHEIQDVSFVPSIHPEFGPGFECFVGGGLSTNPMLAQPLGSWIPLDEVPEVWAGVAGIFRDYGFRRLRNRARLKFLVAQWGIEKFREVLETEYLERKLIDGPVVTTNPGYRDHIGIHPQKDGKFYLGVKPTVGHTTGEQLIAIADVAEKHGITRIRTTAEKELLFLDIERENLTTVARDLDEIGLYSSPSEFRRGIISCTGLEFCKLAHATTKSRAIELVDELEERLGDLDVPIKIALNGCPNSCARTQVSDIGFKGQTVTDADGNRVEGFQVHLGGSMNLDPNFGRKLKGHKVIADEVGEYVTRVVTHFKEQRHEDEHFRDWVQRAAEEDLV

SEQ ID NO: 4 - protein, 262 aa, Lactobacillus acidophilus
MANKVKIGILNLMHDKLDTQSHFIKVLPNADLTFFYPRMHYQNRPIPPEVNMTSEPLDINRVSEFDGFIITGAPIDQIDFSKITYIEEIRYLLQALDNHKIQQLYFCWGAMAALNYFYGIKKKILAEKIFGVFPHLITEPHPLLSGLSQGFMAPHARYAEMDKKQIMQDERLAINAVDDNSHLFMVSAKDNPERNFIFSHIEYGKDSLRDEYNREINAHPERHYKKPINYSMSNPSFQWQDTQKIFFNNWLKKVKDNKLVLN

SEQ ID NO: 5 - protein, 445 aa, Artificial
  Mutated (feedback-deregulated) sequence
MTSASAPSFNPGKGPGSAVGIALLGFGTVGTEVMRLMTEYGDELAHRIGGPLEVRGIAVSDISKPREGVAPELLTEDAFALIEREDVDIVVEVIGGIEYPREVVLAALKAGKSVVTANKALVAAHSAELADAAEAANVDLYFEAAVAGAIPVVGPLRRSLAGDQIQSVMGIVNGTTNFILDAMDSTGADYADSLAEATRLGYAEADPTADVEGHDAASKAAILASIAFHTRVTADDVYCEGISNISAADIEAAQQAGHTIKLLAICEKFTNKEGKSAISARVHPTLLPVSHPLASVNKSFNAIFVEAEAAGRLMFYGNGAGGAPTASAVLGDVVGAARNKVHGGRAPGESTYANLPIADFGETTTRYHLDMDVEDRVEVLAELASLFSEQGISLRTIRQEERDDDARLIVVTHSALESDLSRTVELLKAKPVVKAINSVIRLERD

SEQ ID NO: 6 - protein, 427 aa, Lactobacillus collinoides
MTDTSKLHFETKQVHAGQVVDETGARAVPIYQTTSYVFKDAAQAAGRFGLTDPGNIYTRLTNPTTDVLEKRVAQLENGTAGVALATGAAAVTAAIENVANAGDNIVSASTLYGGTYDLFSVTLPKLGITTTFVDPDDPQNFAKAIDDKTKALYIETIGNPGINIIDIEAVAKIAHDNGIVLIADNTFGTPYLIQPLDHGVDVVIHSATKFIGGHGTTMGGVIVENGKFDYAKSGKYPDFTTPDDTYNGIVWNDINPATFTTKVRAQTLRDTGATISPFNSFLLLQGLESLSLRVERHVSNAQKIAKFLNDHPKVAWVNYPGLPGNKYNDLAKKYFPKGTGSIFTIGLKGGEKAGKDLIEKLNLFSLLANVGDAKSLIIHPASTTHAQLNEEQLKETGITPDLIRLSIGIENVDDLIADLSQALDQID

SEQ ID NO: 7 - protein, 304 aa, Corynebacterium glutamicum
MTTTVASELSPHLKDLENESIHILREVAGQFDKVGLLFSGGKDSVVVYELARRAFAPANVPFELLHVDTGHNFPEVLEFRDNLVERTGARLRVAKVQDWIDRGDLQERPDGTRNPLQTVPLVETIAEQGYDAVLGGARRDEERARAKERVFSVRDSFGGWDPRRQRPELWTLYNGGHLPGENIRVFPISNWTEADIWEYIGARGIELPPIYFSHDREVFERDGMWLTAGEWGGPKKGEEIVTKTVRYRTVGDMSCTGAVLSEARTIDDVIEEIATSTLTERGATRADDRLSESAMEDRKKEGYF

SEQ ID NO: 8 - DNA, 912 bp, Corynebacterium glutamicum
atgaccacaaccgttgcatcagaactttccccacaccttaaagatcttgaaaatgaatccatccacatcctccgcgaggtagctggccagtttgataaggtcggcctgctgttttccggcggtaaggattccgtcgtggtgtacgagcttgcgcgccgcgctttcgctccagctaacgtgccttttgaattgctgcacgtggacaccggccacaacttcccagaggttttggaattccgcgacaacctggtggagcgcaccggcgcccgcctgcgcgtagctaaagtccaggactggatcgatcgcggtgacctgcaggaacgcccagacggcacccgcaacccactgcagactgtccctttggtggagaccatcgctgagcagggctacgacgccgtgcttggtggcgctcgccgcgatgaggagcgtgcccgcgccaaggagcgtgtgttctctgtgcgtgactccttcggtggttgggatccacgccgtcagcgcccagagctgtggaccctctacaacggtggccacctgccaggcgaaaacatccgtgttttcccaatctccaactggactgaagctgacatctgggagtacatcggcgcccgtggcatcgaacttccaccgatctacttctcccacgaccgcgaagttttcgagcgcgacggcatgtggctgaccgcaggcgagtggggtggaccaaagaagggcgaggagatcgtcaccaagactgttcgctaccgcaccgtcggcgatatgtcctgcaccggtgctgtgctctcagaagcccgcaccattgacgatgtgatcgaagagatcgccacctccacccttaccgaacgtggcgcaacccgcgccgatgaccgcctcagcgaatccgcaatggaagaccgcaagaaggaaggctacttc

SEQ ID NO: 9 - DNA, 783 bp, Corynebacterium glutamicum
atgagctttcaactagttaacgccctgaaaaatactggttcggtaaaagatcccgagatctcacccgaaggacctcgcacgaccacaccgttgtcaccagaggtagcaaaacacaacgaggaactcgtcgaaaagcatgctgctgcgttgtatgacgccagcgcgcaagagatcctggaatggacagccgagcacacgccgggcgctattgcagtgaccttgagcatggaaaacaccgtactggcggagctggctgcgcggcacctgccggaagctgatttcctctttttggacaccggttaccacttcaaggaaactcttgaagttgcccgccaggtagatgagcgttattcccagaagcttgtcaccgcgctgccaatcctcaagcgcacggagcaggattccatttatggtctcaacctgtaccgcagcaacccagcggcgtgctgccgaatgcgcaaagttgaaccgctggcggcgtcgttaagcccatacgctggctggatcaccggcctgcgccgcgctgatggcccaacccgtgctcaagcccctgcgctgagcttggatgccaccggcaggctcaagatttctccaattatcacctggtcattggaggaaaccaacgagttcattgcggacaacaacctcatcgatcacccacttacccatcagggttatccatcaattggatgcgaaacctgcacccttcctgttgctgaaggacaagaccctagggccggccgttgggctggaaacgccaagacagaatgcggacttcactca

SEQ ID NO: 10 - DNA, 1683 bp, Corynebacterium glutamicum
atgacaacaaccaccggaagtgcccggccagcacgtgccgccaggaagcctaagcccgaaggccaatggaaaatcgacggcaccgagccgcttaaccatgccgaggaaattaagcaagaagaacccgcttttgctgtcaagcagcgggtcattgatatttactccaagcagggtttttcttccattgcaccggatgacattgccccacgctttaagtggttgggcatttacacccagcgtaagcaggatctgggcggtgaactgaccggtcagcttcctgatgatgagctgcaggatgagtacttcatgatgcgtgtgcgttttgatggcggactggcttcccctgagcgcctgcgtgccgtgggtgaaatttctagggattatgctcgttccaccgcggacttcaccgaccgccagaacattcagctgcactggattcgtattgaagatgtgcctgcgatctgggagaagctagaaaccgtcggactgtccaccatgcttggttgcggtgacgttccacgtgttatcttgggctccccagtttctggcgtagctgctgaagagctgatcgatgccaccccggctatcgatgcgattcgtgagcgctacctagacaaggaagagttccacaaccttcctcgtaagtttaagactgctatcactggcaaccagcgccaggatgttacccacgaaatccaggacgtttccttcgttccttcgattcacccagaattcggcccaggatttgagtgctttgtgggcggcggcctgtccaccaacccaatgcttgctcagccacttggttcttggattccacttgatgaggttccagaagtgtgggctggcgtcgccggaattttccgcgactacggcttccgacgcctgcgtaaccgtgctcgcctcaagttcttggtggcacagtggggtattgagaagttccgtgaagttcttgagaccgaatacctcgagcgcaagctgattgatggcccagttgttaccaccaaccctggctaccgtgaccacattggcattcacccacaaaaggacggcaagttctacctcggtgtgaagccaaccgttggacacaccaccggtgagcagctcattgccattgctgatgttgcagaaaagcacggcatcaccaggattcgtaccacggcggaaaaggaactgctcttcctcgatattgagcgagagaaccttactaccgttgcacgtgacctggatgaaatcggactgtactcttcaccttccgagttccgccgcggcatcatttcctgcaccggcttggagttctgcaagcttgcgcacgcaaccaccaagtcacgagcaattgagcttgtggacgaactggaagagcgactcggcgatttggatgttcccatcaagattgccctgaacggttgccctaactcttgtgcacgcacccaggtttccgacatcggattcaagggacagaccgtcactgatgctgacggcaaccgcgttgaaggtttccaggttcacctgggcggttccatgaacttggatccaaacttcggacgcaagctcaagggccacaaggttattgccgatgaagtgggagagtacgtcactcgcgttgttacccacttcaaggaacagcgccacgaggacgagcacttccgcgattgggtccagcgggccgctgaggaagatttggtg

SEQ ID NO: 11 - protein, 445 aa, Corynebacterium glutamicum; identical to SEQ ID NO: 5

(19) United States
(12) Patent Application Publication
(10) Pub. No.: US 2022/0162655 A1
(43) Pub. Date: May 26, 2022

(54) ENGINEERED BIOSYNTHETIC PATHWAYS FOR PRODUCTION OF L-HOMOCYSTEINE BY FERMENTATION

(71) Applicant: Zymergen Inc., Emeryville, CA (US)

(72) Inventors: Michael Shareef Siddiqui, Oakland, CA (US); Alexander Glennon Shearer, San Francisco, CA (US); Franklin Lu, Emeryville, CA (US); Stefan de Kok, Emeryville, CA (US); Cara Ann Tracewell, Walnut Creek, CA (US); Steven M. Edgar, Albany, CA (US); Jennifer Yip, Emeryville, CA (US)

(73) Assignee: Zymergen Inc., Emeryville, CA (US)

(21) Appl. No.: 17/442,090

(22) PCT Filed: Mar. 24, 2020
(86) PCT No.: PCT/US2020/024512

§ 371 Date: Sep. 22, 2021

Related U.S. Application Data
(60) Provisional application No. 62/824,220, filed on Mar. 26, 2019.

(51) Int. Cl. C12P 13/12; C12N 9/02; C12N 9/12; C12N 15/77

(52) U.S. Cl. CPC C12P 13/12; C12N 9/0051; C12N 9/1241; C12Y 207/07004; C12Y 108/01002; C12Y 108/02001; C12Y 108/04008; C12N 15/77

(57) ABSTRACT

The present disclosure describes the engineering of microbial cells for fermentative production of L-homocysteine and provides novel engineered microbial cells and cultures, as well as related L-homocysteine production methods.

### CROSS-REFERENCE TO RELATED APPLICATIONS

This application claims the benefit of U.S. provisional application No. 62/824,220, filed Mar. 26, 2019, which is hereby incorporated by reference in its entirety.

### STATEMENT AS TO RIGHTS TO INVENTIONS MADE UNDER FEDERALLY SPONSORED RESEARCH AND DEVELOPMENT

This invention was made with Government support under Agreement No. HR0011-15-9-0014, awarded by DARPA. The Government has certain rights in the invention.

### INCORPORATION BY REFERENCE OF THE SEQUENCE LISTING

This application includes a sequence listing which has been submitted electronically in ASCII format and is hereby incorporated by reference in its entirety. This ASCII copy, created on Mar. 24, 2020, is named ZMGNP023WO_SeqList_ST25.txt. and is 32,589 bytes in size.

### FIELD OF THE DISCLOSURE

The present disclosure relates generally to the area of engineering microbes for production of L-homocysteine by fermentation.

### BACKGROUND

Homocysteine is a non-proteinogenic α-amino acid. It is a homologue of the amino acid cysteine, differing by an additional methylene bridge (—CH₂—). It is produced from methionine by the removal of its terminal C^{ε} methyl group. Homocysteine can be recycled into methionine or converted into cysteine with the aid of certain B-vitamins Homocysteine also acts as an allosteric antagonist at Dopamine D₂ receptors.

### SUMMARY

The disclosure provides engineered microbial cells, cultures of the microbial cells, and methods for the production of L-homocysteine, including the following:

Embodiment 1: An engineered microbial cell that includes increased activity of at least one upstream pathway enzyme leading to L-homocysteine, wherein the at least one upstream pathway enzyme is selected from the group consisting of: (a) 3-phosphoadenosine-5-phosphosulfate sulfotransferase (PAPS reductase); (b) sulfite reductase, and (c) sulfate adenylyltransferase (ATP sulfurase), said increased activity being increased relative to a control cell, wherein the engineered microbial cell produces L-homocysteine.

Embodiment 2: The engineered microbial cell of embodiment 1, wherein the engineered microbial cell expresses at least two of said upstream pathway enzymes, wherein the at least two upstream pathway enzymes are selected from the group consisting of: (a) a 3-phosphoadenosine-5-phosphosulfate sulfotransferase (PAPS reductase) and a sulfite reductase; (b) a sulfite reductase and a sulfate adenylyltransferase (ATP sulfurase); and (c) a 3-phosphoadenosine-5-phosphosulfate sulfotransferase (PAPS reductase) and a sulfate adenylyltransferase (ATP sulfurase).

Embodiment 3: The engineered microbial cell of embodiment 1 or embodiment 2, wherein said upstream pathway enzymes are heterologous enzymes.

Embodiment 4: The engineered microbial cell of embodiment 3, wherein the engineered microbial cell expresses: (a) a heterologous 3-phosphoadenosine-5-phosphosulfate sulfotransferase (PAPS reductase); (b) a heterologous sulfite reductase, and (c) a heterologous sulfate adenylyltransferase (ATP sulfurase).

Embodiment 5: The engineered microbial cell of any one of embodiments 1-4, wherein the engineered microbial cell includes increased activity of one or more additional upstream pathway enzyme(s) leading to L-homocysteine that is/are selected from the group consisting of phosphoadenosine phosphosulfate reductase (PAPS reductase), and homocysteine synthase, said increased activity being increased relative to a control cell.

Embodiment 6: The engineered microbial cell of any one of embodiments 1-5, wherein the engineered microbial cell includes increased activity of a sulfate transporter, said increased activity being increased relative to a control cell.

Embodiment 7: The engineered microbial cell of any one of embodiments 1-6, wherein the engineered microbial cell includes increased activity of one or more upstream pathway enzymes leading to O-acetyl-L-homoserine, said increased activity being increased relative to a control cell.

Embodiment 8: The engineered microbial cell of embodiment 7, wherein the one or more upstream pathway enzymes leading to O-acetyl-L-homoserine is/are selected from the group consisting of phosphoenolpyruvate carboxykinase (PEP carboxykinase), pyruvate kinase, pyruvate carboxylase, glutamate dehydrogenase, aspartate transaminase (aspartate aminotransferase), aspartate kinase (aspartokinase), aspartate-semialdehyde dehydrogenase, homoserine dehydrogenase, and L-homoserine-O-acetyltransferase.

Embodiment 9: The engineered microbial cell of embodiment 8, the one or more upstream pathway enzymes leading to O-acetyl-L-homoserine includes PEP carboxykinase, and the activity of pyruvate carboxylase is reduced relative to a control cell.

Embodiment 10: The engineered microbial cell of any one of embodiments 1-9, wherein the activity of malate dehydrogenase is reduced relative to a control cell.

Embodiment 11: The engineered microbial cell of any one of embodiments 1-10, wherein the activity of the one or more upstream pathway enzymes is increased by expressing one or more feedback-deregulated enzyme(s).

Embodiment 12: The engineered microbial cell of embodiment 11, where the one or more feedback-deregulated enzyme (s) is/are selected from the group consisting of a feedback-deregulated aspartate kinase, a feedback-deregulated homoserine dehydrogenase, a feedback-deregulated aspartate-semialdehyde dehydrogenase, and a feedback-deregulated pyruvate carboxylase.

Embodiment 13: The engineered microbial cell of any one of embodiments 1-10, wherein the activity of the one or more upstream pathway enzymes is increased by expressing one or more upstream pathway enzyme(s) that is/are normally subject to feedback inhibition at the transcriptional level so as to reduce said feedback inhibition at the transcriptional level.

Embodiment 14: The engineered microbial cell of embodiment 13, wherein reduced feedback inhibition at the transcriptional level is achieved by a method including expressing aspartate kinase from a constitutive promoter.

Embodiment 15: The engineered microbial cell of any one of embodiments 1-14, wherein the engineered microbial cell includes reduced activity of one or more enzyme(s) that consume one or more upstream pathway precursors, said reduced activity being reduced relative to a control cell.

Embodiment 16: The engineered microbial cell of embodiment 15, wherein the one or more enzyme(s) that consume one or more upstream pathway precursors is/are selected from the group consisting of cystathionine gamma-synthase, homoserine kinase, and L-homoserine succinyl transferase.

Embodiment 17: The engineered microbial cell of any one of embodiments 1-16, wherein the engineered microbial cell includes reduced activity of one or more enzyme(s) that consume L-homocysteine, said reduced activity being reduced relative to a control cell.

Embodiment 18: The engineered microbial cell of embodiment 17, wherein the one or more enzyme(s) that consume L-homocysteine is/are selected from the group consisting of cystathionine beta-synthase and methionine synthase.

Embodiment 19: The engineered microbial cell of any one of embodiments 15-18, wherein the reduced activity is achieved by one or more means selected from the group consisting of gene deletion, gene disruption, altering regulation of a gene, and replacing a native promoter with a less active promoter.

Embodiment 20: The engineered microbial cell of any one of embodiments 1-19, wherein the engineered microbial cell includes reduced activity of one or more upstream pathway enzymes leading to cysteine, said reduced activity being reduced relative to a control cell.

Embodiment 21: The engineered microbial cell of embodiment 20, wherein the one or more upstream pathway enzymes leading to cysteine is/are selected from the group consisting of 3-phosphoglycerate dehydrogenase, phosphoserine transaminase, phosphoserine phosphatase, serine-O-acetyltransferase, and cysteine synthase.

Embodiment 22: The engineered microbial cell of any of embodiments 1-21, wherein the engineered microbial cell includes altered cofactor specificity of one or more upstream pathway enzyme(s) from the reduced form of nicotinamide adenine dinucleotide phosphate (NADPH) to the reduced from of nicotinamide adenine dinucleotide (NADH).

Embodiment 23: The engineered microbial cell of embodiment 22, wherein the one or more upstream pathway enzyme(s) whose cofactor specificity is altered is/are selected from the group consisting of aspartate semi-aldehyde dehydrogenase, PAPS reductase, and sulfite reductase.

Embodiment 24: An engineered microbial cell that includes means for increasing the activity of at least one upstream pathway enzyme leading to L-homocysteine, wherein the at least one upstream pathway enzyme is selected from the group consisting of: (a) 3-phosphoadenosine-5-phosphosulfate sulfotransferase (PAPS reductase); (b) sulfite reductase, and (c) sulfate adenylyltransferase (ATP sulfurase), said increased activity being increased relative to a control cell, wherein the engineered microbial cell produces L-homocysteine.

Embodiment 25: The engineered microbial cell of embodiment 24, wherein the engineered microbial cell includes means for expressing at least two of said upstream pathway enzymes, wherein the at least two upstream pathway enzymes are selected from the group consisting of: (a) a 3-phosphoadenosine-5-phosphosulfate sulfotransferase (PAPS reductase) and a sulfite reductase; (b) a sulfite reductase and a sulfate adenylyltransferase (ATP sulfurase); and (c) a 3-phosphoadenosine-5-phosphosulfate sulfotransferase (PAPS reductase) and a sulfate adenylyltransferase (ATP sulfurase).

Embodiment 26: The engineered microbial cell of embodiment 24 or embodiment 25, wherein said upstream pathway enzymes are heterologous enzymes.

Embodiment 27: The engineered microbial cell of embodiment 26, wherein the engineered microbial cell includes means for expressing: (a) a heterologous 3-phosphoadenosine-5-phosphosulfate sulfotransferase (PAPS reductase); (b) a heterologous sulfite reductase, and (c) a heterologous sulfate adenylyltransferase (ATP sulfurase).

Embodiment 28: The engineered microbial cell of any one of embodiments 24-27, wherein the engineered microbial cell includes means for increasing the activity of one or more additional upstream pathway enzyme(s) leading to L-homocysteine that is/are selected from the group consisting of phosphoadenosine phosphosulfate reductase (PAPS reductase) and homocysteine synthase, said increased activity being increased relative to a control cell.

Embodiment 29: The engineered microbial cell of any one of embodiments 24-28, wherein the engineered microbial cell includes means for increasing the activity of a sulfate transporter, said increased activity being increased relative to a control cell.

Embodiment 30: The engineered microbial cell of any one of embodiments 24-29, wherein the engineered microbial cell includes means for increasing the activity of one or more upstream pathway enzymes leading to O-acetyl-L-homoserine, said increased activity being increased relative to a control cell.

Embodiment 31: The engineered microbial cell of embodiment 30, wherein the one or more upstream pathway enzymes leading to O-acetyl-L-homoserine is/are selected from the group consisting of phosphoenolpyruvate carboxykinase (PEP carboxykinase), pyruvate kinase, pyruvate carboxylase, glutamate dehydrogenase, aspartate transaminase (aspartate aminotransferase), aspartate kinase (aspartokinase), aspartate-semialdehyde dehydrogenase, homoserine dehydrogenase, and L-homoserine-O-acetyltransferase.

Embodiment 32: The engineered microbial cell of embodiment 31, the one or more upstream pathway enzymes leading to O-acetyl-L-homoserine includes PEP carboxykinase, and the engineered microbial cell includes means for reducing the activity of pyruvate carboxylase relative to a control cell.

Embodiment 33: The engineered microbial cell of any one of embodiments 24-32, wherein the engineered microbial cell includes means for reducing the activity of malate dehydrogenase relative to a control cell.

Embodiment 34: The engineered microbial cell of any one of embodiments 24-33, wherein the engineered microbial cell includes means for expressing one or more feedback-deregulated enzyme(s).

Embodiment 35: The engineered microbial cell of embodiment 34, where the one or more feedback-deregulated enzyme (s) is/are selected from the group consisting of a feedback-deregulated aspartate kinase, a feedback-deregulated homoserine dehydrogenase, a feedback-deregulated aspartate-semialdehyde dehydrogenase, and a feedback-deregulated pyruvate carboxylase.

Embodiment 36: The engineered microbial cell of any one of embodiments 24-33, wherein the activity of the one or more upstream pathway enzymes is increased by expressing one or more upstream pathway enzyme(s) that is/are normally subject to feedback inhibition at the transcriptional level so as to reduce said feedback inhibition at the transcriptional level.

Embodiment 37: The engineered microbial cell of embodiment 36, wherein reduced feedback inhibition at the transcriptional level is achieved by a method including expressing aspartate kinase from a constitutive promoter.

Embodiment 38: The engineered microbial cell of any one of embodiments 24-37, wherein the engineered microbial cell includes means for reducing the activity of one or more enzyme(s) that consume one or more upstream pathway precursors, said reduced activity being reduced relative to a control cell.

Embodiment 39: The engineered microbial cell of embodiment 38, wherein the one or more enzyme(s) that consume one or more upstream pathway precursors is/are selected from the group consisting of cystathionine gamma-synthase, homoserine kinase, and L-homoserine succinyl transferase.

Embodiment 40: The engineered microbial cell of any one of embodiments 24-39, wherein the engineered microbial cell includes means for reducing the activity of one or more enzyme(s) that consume(s) L-homocysteine, said reduced activity being reduced relative to a control cell.

Embodiment 41: The engineered microbial cell of embodiment 40, wherein the one or more enzyme(s) that consume L-homocysteine is/are selected from the group consisting of cystathionine beta-synthase and methionine synthase.

Embodiment 42: The engineered microbial cell of any one of embodiments 24-41, wherein the engineered microbial cell includes means for reducing the activity of one or more upstream pathway enzymes leading to cysteine, said reduced activity being reduced relative to a control cell.

Embodiment 43: The engineered microbial cell of embodiment 42, wherein the one or more upstream pathway enzymes leading to cysteine is/are selected from the group consisting of 3-phosphoglycerate dehydrogenase, phosphoserine transaminase, phosphoserine phosphatase, serine-O-acetyltransferase, and cysteine synthase.

Embodiment 44: The engineered microbial cell of any of embodiments 24-43, wherein the engineered microbial cell includes means for altering the cofactor specificity of one or more upstream pathway enzyme(s) from the reduced form of nicotinamide adenine dinucleotide phosphate (NADPH) to the reduced from of nicotinamide adenine dinucleotide (NADH).

Embodiment 45: The engineered microbial cell of embodiment 44, wherein the one or more upstream pathway enzyme(s) whose cofactor specificity is altered is/are selected from the group consisting of aspartate semi-aldehyde dehydrogenase, PAPS reductase, and sulfite reductase.

Embodiment 46: The engineered microbial cell of any one of embodiments 1-45, wherein the engineered microbial cell is a bacterial cell.

Embodiment 47: The engineered microbial cell of embodiment 46, wherein the bacterial cell is a cell of the genus Corynebacteria.

Embodiment 48: The engineered microbial cell of embodiment 47, wherein the bacterial cell is a cell of the species glutamicum.

Embodiment 49: The engineered microbial cell of embodiment 48, wherein the engineered microbial cell includes a heterologous 3-phosphoadenosine-5-phosphposulfate sulfotransferase (PAPS reductase) having at least 70% amino acid sequence identity with a Corynebacteria glutamicum 3-phosphoadenosine-5-phosphposulfate sulfotransferase (PAPS reductase) including SEQ ID NO:2.

Embodiment 50: The engineered microbial cell of embodiment 48 or embodiment 49, wherein the engineered microbial cell additionally includes a heterologous sulfite reductase having at least 70% amino acid sequence identity with a Corynebacteria glutamicum sulfite reductase hemoprotein beta-component including SEQ ID NO:3.

Embodiment 51: The engineered microbial cell of any one of embodiments 48-50, wherein the engineered microbial cell additionally includes a heterologous sulfate adenylyltransferase (ATP sulfurase) having at least 70% amino acid sequence identity with a Corynebacteria glutamicum sulfate adenylyltransferase subunit 1 including SEQ ID NO:1.

Embodiment 52: The engineered microbial cell of embodiment 51, wherein the engineered microbial cell is a Corynebacteria glutamicum cell that expresses: (a) a heterologous Corynebacteria glutamicum 3-phosphoadenosine-5-phosphposulfate sulfotransferase (PAPS reductase) including SEQ ID NO:2; (b) a heterologous Corynebacteria glutamicum sulfite reductase hemoprotein beta-component including SEQ ID NO:3; and (c) a heterologous Corynebacteria glutamicum sulfate adenylyltransferase subunit 1 including SEQ ID NO:1.

Embodiment 52-1: The engineered microbial cell of embodiment 51, wherein the engineered microbial cell is a Corynebacteria glutamicum cell that expresses: (a) a heterologous Corynebacteria glutamicum 3-phosphoadenosine-5-phosphposulfate sulfotransferase (PAPS reductase) including SEQ ID NO:2; (b) a heterologous Corynebacteria glutamicum sulfite reductase hemoprotein beta-component including SEQ ID NO:3; and (c) a heterologous Corynebacteria glutamicum sulfate adenylyltransferase including SEQ ID NO:7.

Embodiment 52-2: The engineered microbial cell of embodiment 52-1, wherein the engineered microbial cell additionally expresses: (a) a heterologous Lactobacillus acidophilus serine O-acetyltransferase comprising SEQ ID NO:4; (b) a heterologous Corynebacteria glutamicum homoserine dehydrogenase comprising SEQ ID NO:11; and (c) a heterologous Lactobacillus collinoides O-acetylhomoserine aminocarboxypropyltransferase comprising SEQ ID NO:6.

Embodiment 53: The engineered microbial cell of any one of embodiments 1-52, wherein, when cultured, the engineered microbial cell produces L-homocysteine at a level of at least 5 mg/L of culture medium.

Embodiment 54: The engineered microbial cell of embodiment 53, wherein, when cultured, the engineered microbial cell produces L-homocysteine at a level of at least 15 mg/L of culture medium.

Embodiment 55: A culture of engineered microbial cells according to any one of embodiments 1-54.

Embodiment 56: The culture of embodiment 55, wherein the culture includes a sulfur source that is in a reduced form, relative to sulfate.

Embodiment 57: The culture of embodiment 56, wherein the sulfur source includes a sulfur source selected from the group consisting of a sulfide, a thiosulfate, a methylsulfonate, an ametryne, a prometryne, and any combination thereof.

Embodiment 58: The culture of any one of embodiments 55-57, wherein the substrate includes a carbon source and a nitrogen source selected from the group consisting of urea, an ammonium salt, ammonia, and any combination thereof.

Embodiment 59: The culture of any one of embodiments 55-58, wherein the engineered microbial cells are present in a concentration such that the culture has an optical density at 600 nm of 10-500.

Embodiment 60: The culture of any one of embodiments 55-59, wherein the culture includes L-homocysteine.

Embodiment 61: The culture of any one of embodiments 55-60, wherein the culture includes L-homocysteine at a level of at least 15 mg/L of culture medium.

Embodiment 62: A method of culturing engineered microbial cells according to any one of embodiments 1-54, the method including culturing the cells under conditions suitable for producing L-homocysteine.

Embodiment 63: The method of embodiment 62, wherein the method includes culturing the engineered microbial cells in the presence of a sulfur source that is in a reduced form, relative to sulfate.

Embodiment 64: The culture of embodiment 63, wherein the sulfur source includes a sulfur source selected from the group consisting of a sulfide, a thiosulfate, a methylsulfonate, an ametryne, a prometryne, and any combination thereof.

Embodiment 65: The method of any one of embodiments 62-64, wherein the method includes fed-batch culture, with an initial glucose level in the range of 1-100 g/L, followed by controlled sugar feeding.

Embodiment 66: The method of any one of embodiments 62-65, wherein the fermentation substrate includes glucose and a nitrogen source selected from the group consisting of urea, an ammonium salt, ammonia, and any combination thereof.

Embodiment 67: The method of any one of embodiments 62-66, wherein the culture is pH-controlled during culturing.

Embodiment 68: The method of any one of embodiments 62-67, wherein the culture is aerated during culturing.

Embodiment 69: The method of any one of embodiments 62-68, wherein the engineered microbial cells produce L-homocysteine at a level of at least 15 mg/L of culture medium.

Embodiment 70: The method of any one of embodiments 62-69, wherein the method additionally includes recovering L-homocysteine from the culture.

### BRIEF DESCRIPTION OF THE DRAWINGS

FIG. 1: Biosynthetic pathway for L-homocysteine.

FIG. 2: Fermentation processes for producing L-homocysteine in engineered strains of Corynebacteria glutamicum. (See also Example 1.)

FIG. 3: Integration of Promoter-Gene-Terminator into Saccharomyces cerevisiae and Yarrowia lipolytica.

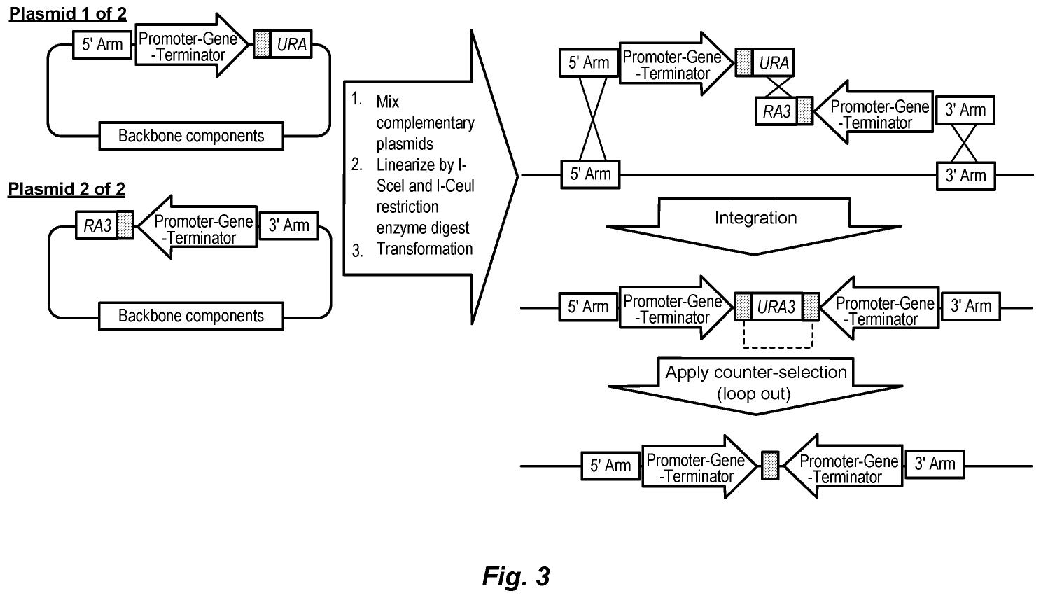

FIG. 4: Promoter replacement in Saccharomyces cerevisiae and Yarrowia lipolytica.

FIG. 5: Targeted gene deletion in Saccharomyces cerevisiae and Yarrowia lipolytica.

FIG. 6: Integration of Promoter-Gene-Terminator into Corynebacteria glutamicum and Bacillus subtilis.

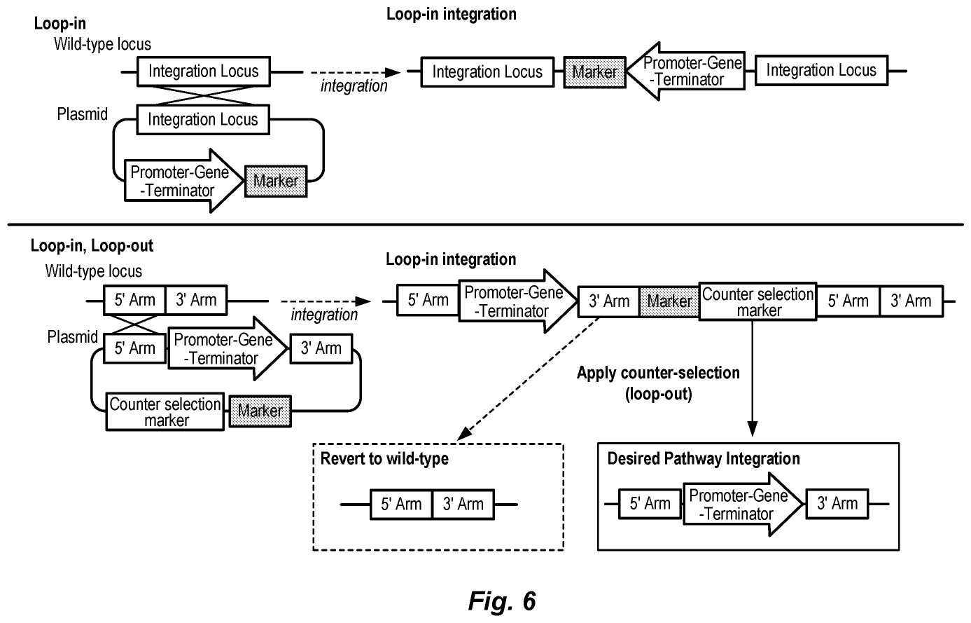

### DETAILED DESCRIPTION

This disclosure describes a method for the production of the small molecule L-homocysteine via fermentation by a microbial host from simple carbon and nitrogen sources, such as glucose and urea, respectively. In the work described herein, a titer of about 18 mg/L L-homocysteine was achieved in engineered Corynebacteria glutamicum.

### Definitions

Terms used in the claims and specification are defined as set forth below unless otherwise specified.

The term “fermentation” is used herein to refer to a process whereby a microbial cell converts one or more substrate(s) into a desired product (such as L-homocysteine) by means of one or more biological conversion steps, without the need for any chemical conversion step.

The term “engineered” is used herein, with reference to a cell, to indicate that the cell contains at least one targeted genetic alteration introduced by man that distinguishes the engineered cell from the naturally occurring cell.

The term “native” is used herein to refer to a cellular component, such as a polynucleotide or polypeptide, that is naturally present in a particular cell. A native polynucleotide or polypeptide is endogenous to the cell.

When used with reference to a polynucleotide or polypeptide, the term “non-native” refers to a polynucleotide or polypeptide that is not naturally present in a particular cell.

When used with reference to the context in which a gene is expressed, the term “non-native” refers to a gene expressed in any context other than the genomic and cellular context in which it is naturally expressed. A gene expressed in a non-native manner may have the same nucleotide sequence as the corresponding gene in a host cell, but may be expressed from a vector or from an integration point in the genome that differs from the locus of the native gene.

The term “heterologous” is used herein to describe a polynucleotide or polypeptide introduced into a host cell. This term encompasses a polynucleotide or polypeptide, respectively, derived from a different organism, species, or strain than that of the host cell. In this case, the heterologous polynucleotide or polypeptide has a sequence that is different from any sequence(s) found in the same host cell. However, the term also encompasses a polynucleotide or polypeptide that has a sequence that is the same as a sequence found in the host cell, wherein the polynucleotide or polypeptide is present in a different context than the native sequence (e.g., a heterologous polynucleotide can be linked to a different promotor and inserted into a different genomic location than that of the native sequence). “Heterologous expression” thus encompasses expression of a sequence that is non-native to the host cell, as well as expression of a sequence that is native to the host cell in a non-native context.

As used with reference to polynucleotides or polypeptides, the term “wild-type” refers to any polynucleotide having a nucleotide sequence, or polypeptide having an amino acid, sequence present in a polynucleotide or polypeptide from a naturally occurring organism, regardless of the source of the molecule; i.e., the term “wild-type” refers to sequence characteristics, regardless of whether the molecule is purified from a natural source; expressed recombinantly, followed by purification; or synthesized. The term “wild-type” is also used to denote naturally occurring cells.

A “control cell” is a cell that is otherwise identical to an engineered cell being tested, including being of the same genus and species as the engineered cell, but lacks the specific genetic modification(s) being tested in the engineered cell.

Enzymes are identified herein by the reactions they catalyze and, unless otherwise indicated, refer to any polypeptide capable of catalyzing the identified reaction. Unless otherwise indicated, enzymes may be derived from any organism and may have a native or mutated amino acid sequence. As is well known, enzymes may have multiple functions and/or multiple names, sometimes depending on the source organism from which they derive. The enzyme names used herein encompass orthologs, including enzymes that may have one or more additional functions or a different name.

The term “feedback-deregulated” is used herein with reference to an enzyme that is normally negatively regulated by a downstream product of the enzymatic pathway (i.e., feedback-inhibition) in a particular cell. In this context, a “feedback-deregulated” enzyme is a form of the enzyme that is less sensitive to feedback-inhibition than the enzyme native to the cell or a form of the enzyme that is native to the cell, but is naturally less sensitive to feedback inhibition than one or more other natural forms of the enzyme. A feedback-deregulated enzyme may be produced by introducing one or more mutations into a native enzyme. Alternatively, a feedback-deregulated enzyme may simply be a heterologous, native enzyme that, when introduced into a particular microbial cell, is not as sensitive to feedback-inhibition as the native, native enzyme. In some embodiments, the feedback-deregulated enzyme shows no feedback-inhibition in the microbial cell.

The term “L-homocysteine” refers to a chemical compound of the formula C₄H₉NO₂S also known as “2-amino-4-sulfanylbutanoic acid” (CAS #6027-13-0 [L-isomer]).

The term “sequence identity,” in the context of two or more amino acid or nucleotide sequences, refers to two or more sequences that are the same or have a specified percentage of amino acid residues or nucleotides that are the same, when compared and aligned for maximum correspondence, as measured using a sequence comparison algorithm or by visual inspection.

For sequence comparison to determine percent nucleotide or amino acid sequence identity, typically one sequence acts as a “reference sequence,” to which a “test” sequence is compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence relative to the reference sequence, based on the designated program parameters. Alignment of sequences for comparison can be conducted using BLAST set to default parameters.

The term “titer,” as used herein, refers to the mass of a product (e.g., L-homocysteine) produced by a culture of microbial cells divided by the culture volume.

As used herein with respect to recovering L-homocysteine from a cell culture, “recovering” refers to separating the L-homocysteine from at least one other component of the cell culture medium.

### Engineering Microbes for L-Homocysteine Production

L-Homocysteine Biosynthesis Pathway

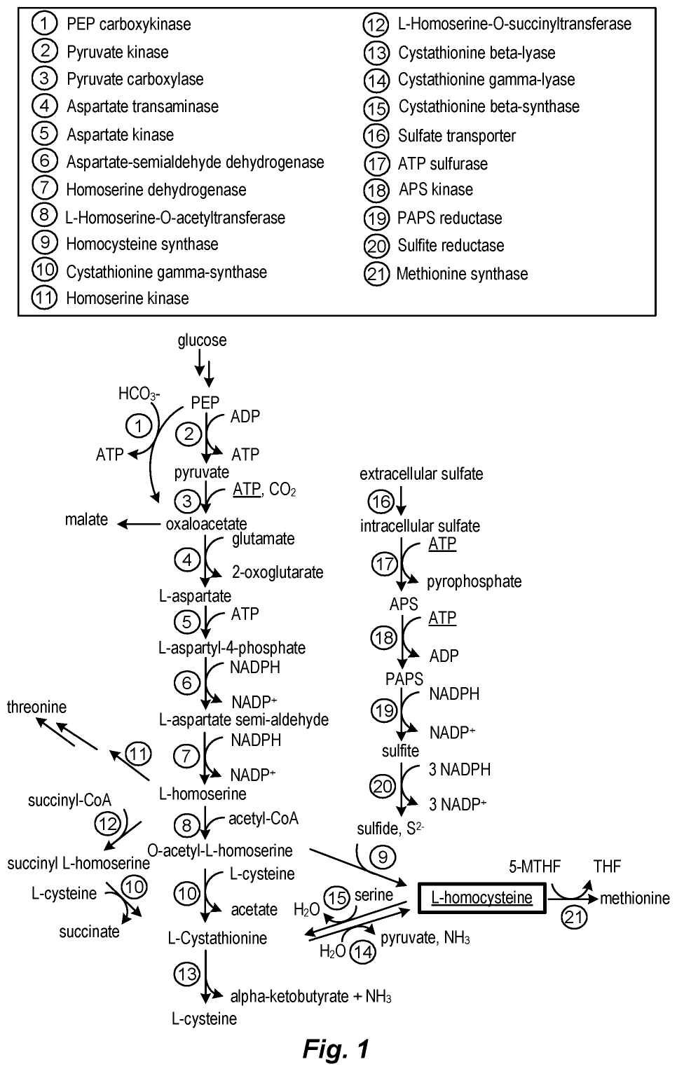

L-homocysteine can be produced in one enzymatic step, requiring either the enzyme L-homocysteine synthase or the enzyme cystathionine gamma-lyase. The L-homocysteine biosynthesis pathway is shown in FIG. 1. Not all microbes have these enzymes. For example, homocysteine synthases have been identified in Corynebacteria glutamicum and Saccharomyces cerevisiae, whereas this enzyme is absent in Bacillus subtilis. Cystathionine gamma-lyases are naturally present in B. subtilis, S. cerevisiae, and Y. lipolytica, but absent in C. glutamicum. L-homocysteine production can be enabled in a host that does not naturally produce it, or potentially improved in one that does, by the addition of one or more L-homocysteine synthases and/or one or more cystathionine gamma-lyases.

Engineering for Microbial L-Homocysteine Production

Any L-homocysteine synthase and/or cystathionine gamma-lyase that is active in the microbial cell being engineered may be introduced into the cell, typically by introducing and expressing the gene(s) encoding the enzyme(s)s using standard genetic engineering techniques. Suitable L-homocysteine beta-synthases and/or cystathionine gamma-lyases may be derived from any source, including plant, archaeal, fungal, gram-positive bacterial, and gram-negative bacterial sources.

One or more copies of any of these genes can be introduced into a selected microbial host cell. If more than one copy of a gene is introduced, the copies can have the same or different nucleotide sequences. In some embodiments, one or both (or all) of the heterologous gene(s) is/are expressed from a strong, constitutive promoter. In some embodiments, the heterologous gene(s) is/are expressed from an inducible promoter. The heterologous gene(s) can optionally be codon-optimized to enhance expression in the selected microbial host cell. The codon-optimization tables used in the Examples are as follows: Bacillus subtilis Kazusa codon table: www.kazusa.or.jp/codon/cgi-bin/showcodon.cgi?species=1423&aa=1&style=N; Yarrowia lipolytica Kazusa codon table: www.kazusa.or.jp/codon/cgi-bin/showcodon.cgi?species=4952&aa=1&style=N; Corynebacteria glutamicum Kazusa codon table: www.kazusa.or.jp/codon/cgi-bin/showcodon.cgi?species=340322&aa=1&style=N; Saccharomyces cerevisiae Kazusa codon table: www.kazusa.or.jp/codon/cgi-bin/showcodon.cgi?species=4932&aa=1&style=N. Also used, was a modified, combined codon usage scheme for S. cereviae and C. glutamicum, which is reproduced below.

Modified Codon Usage Table for Sc and Cg

| Amino Acid | Codon | Fraction |
|---|---|---|
| A | GCG | 0.22 |
| A | GCA | 0.29 |
| A | GCT | 0.24 |
| A | GCC | 0.25 |
| C | TGT | 0.36 |
| C | TGC | 0.64 |
| D | GAT | 0.56 |
| D | GAC | 0.44 |
| E | GAG | 0.44 |
| E | GAA | 0.56 |
| F | TTT | 0.37 |
| F | TTC | 0.63 |
| G | GGG | 0.08 |
| G | GGA | 0.19 |
| G | GGT | 0.3 |
| G | GGC | 0.43 |
| H | CAT | 0.32 |
| H | CAC | 0.68 |
| I | ATA | 0.03 |
| I | ATT | 0.38 |
| I | ATC | 0.59 |
| K | AAG | 0.6 |
| K | AAA | 0.4 |
| L | TTG | 0.29 |
| L | TTA | 0.05 |
| L | CTG | 0.29 |
| L | CTA | 0.06 |
| L | CTT | 0.17 |
| L | CTC | 0.14 |
| M | ATG | 1 |
| N | AAT | 0.33 |
| N | AAC | 0.67 |
| P | CCG | 0.22 |
| P | CCA | 0.35 |
| P | CCT | 0.23 |
| P | CCC | 0.2 |
| Q | CAG | 0.61 |
| Q | CAA | 0.39 |
| R | AGG | 0.11 |
| R | AGA | 0.12 |
| R | CGG | 0.09 |
| R | CGA | 0.17 |
| R | CGT | 0.34 |
| R | CGC | 0.18 |
| S | AGT | 0.08 |
| S | AGC | 0.16 |
| S | TCG | 0.12 |
| S | TCA | 0.13 |
| S | TCT | 0.17 |
| S | TCC | 0.34 |
| T | ACG | 0.14 |
| T | ACA | 0.12 |
| T | ACT | 0.2 |
| T | ACC | 0.53 |
| V | GTG | 0.36 |
| V | GTA | 0.1 |
| V | GTT | 0.26 |
| V | GTC | 0.28 |
| W | TGG | 1 |
| Y | TAT | 0.34 |
| Y | TAC | 0.66 |

Increasing the Activity of Upstream Enzymes

One approach to increasing L-homocysteine production in a microbial cell that is capable of such production is to increase the activity of one or more upstream enzymes in the L-homocysteine biosynthesis pathway. Upstream pathway enzymes include all enzymes involved in the conversions from a feedstock all the way to a metabolite that can be directly converted to L-homocysteine (e.g., O-acetyl-L-homoserine or L-cystathionine). Illustrative enzymes, for this purpose, include, but are not limited to, those shown in FIG. 1 in the pathways leading to these metabolites. Suitable upstream pathway genes encoding these enzymes may be derived from any available source, including, for example, those disclosed herein.

In some embodiments, the activity of one or more upstream pathway enzymes is increased by modulating the expression or activity of the native enzyme(s). For example, native regulators of the expression or activity of such enzymes can be exploited to increase the activity of suitable enzymes.

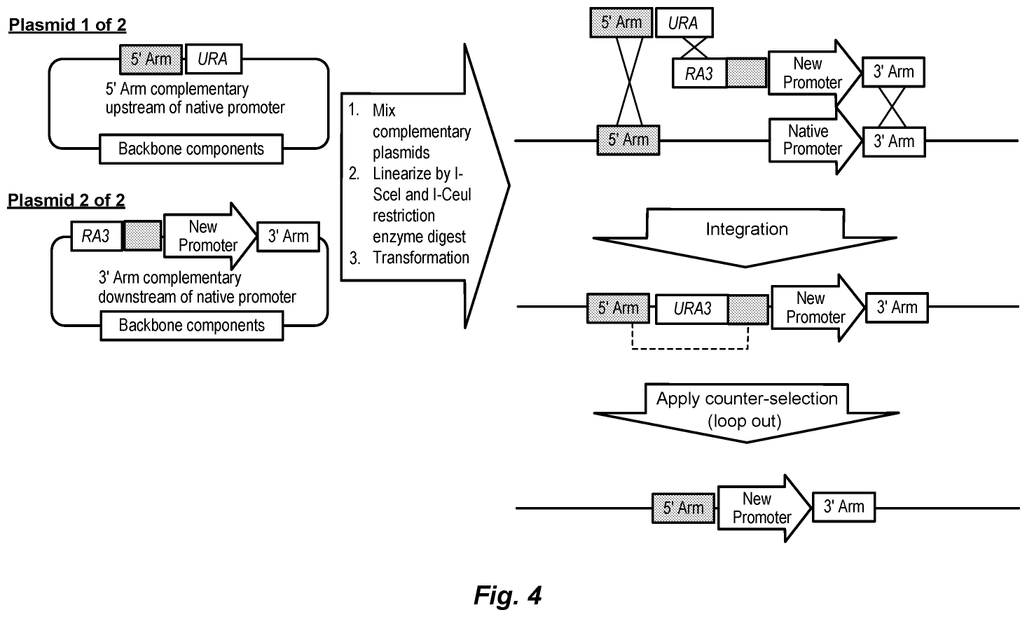

Alternatively, or in addition, one or more promoters can be substituted for native promoters using, for example, a technique such as that illustrated in FIG. 4. In certain embodiments, the replacement promoter is stronger than the native promoter and/or is a constitutive promoter.

In some embodiments, the activity of one or more upstream pathway enzymes is supplemented by introducing one or more of the corresponding genes into the engineered microbial host cell. An introduced upstream pathway gene may be from an organism other than that of the host cell or may simply be an additional copy of a native gene. In some embodiments, one or more such genes are introduced into a microbial host cell capable of L-homocysteine production and expressed from a strong constitutive promoter and/or can optionally be codon-optimized to enhance expression in the selected microbial host cell.

For upstream pathway enzymes that are normally subject to feedback inhibition at the transcriptional level, enzyme activity can be upregulated by blocking or bypassing the normal feedback inhibition. In certain embodiments, a recombinant construct in which the native promoter for this gene is replaced with a strong constitutive promoter can be introduced into an engineered microbial cell to produce more of the enzyme under conditions where expression of the enzyme would normally be inhibited. LysC, for example, encodes aspartate kinase and is feedback-inhibited at the transcriptional level.

In various embodiments, the engineering of a L-homocysteine-producing microbial cell to increase the activity of one or more upstream pathway enzymes increases the L-homocysteine titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in L-homocysteine titer is in the range of 10-fold to 1000-fold, 20-fold to 500-fold, 50-fold to 400-fold, 10-fold to 300-fold, or any range bounded by any of the values listed above. (Ranges herein include their endpoints.) These increases are determined relative to the L-homocysteine titer observed in a L-homocysteine-producing microbial cell that lacks any increase in activity of upstream pathway enzymes. This reference cell may have one or more other genetic alterations aimed at increasing L-homocysteine production.

In various embodiments, the L-homocysteine titers achieved by increasing the activity of one or more upstream pathway enzymes are at least 10, 20, 30, 40, 50, 75, 100, 200, 300, 400, 500, 600, 700, 800, or 900 μg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 mg/L. In various embodiments, the titer is in the range of 50 μg/L to 100 mg/L, 75 μg/L to 75 mg/L, 100 μg/L to 50 mg/L, 200 μg/L to 40 gm/L, 300 μg/L to 30 gm/L, 500 μg/L to 25 mg/L, 1 mg/L to 20 mg/L, or any range bounded by any of the values listed above.

In Corynebacteria glutamicum, for example, an about 18 mg/L titer of L-homocysteine was achieved by overexpressing: a sulfate adenylyltransferase subunit 1 gene (also called “ATP sulfurylase,” enzyme 17 in FIG. 1) from C. glutamicum, a 3-phosphoadenosine-5-phosphposulfate sulfotransferase gene (also called “PAPS reductase,” enzyme 19 in FIG. 1) from C. glutamicum, and a sulfite reductase hemoprotein beta-component gene (also called “sulfite reductase,” enzyme 20 in FIG. 1) from C. glutamicum (see Example 1).

Feedback-Deregulated Enzymes

Another approach to increasing L-homocysteine production in a microbial cell engineered for enhanced L-homocysteine production is to introduce feedback-deregulated forms of one or more enzymes that are normally subject to feedback regulation (e.g., those discussed above in the Summary) A feedback-deregulated form can be a heterologous, native enzyme that is less sensitive to feedback inhibition than the native enzyme in the particular microbial host cell. Alternatively, a feedback-deregulated form can be a variant of a native or heterologous enzyme that has one or more mutations or truncations rendering it less sensitive to feedback inhibition than the corresponding native enzyme.

In some embodiments, the feedback-deregulated enzyme need not be “introduced,” in the traditional sense. Rather, the microbial host cell selected for engineering can be one that has a native enzyme that is naturally insensitive to feedback inhibition.

In various embodiments, the engineering of a L-homocysteine-producing microbial cell to include one or more feedback-regulated enzymes increases the L-homocysteine titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in L-homocysteine titer is in the range of 10-fold to 1000-fold, 20-fold to 500-fold, 50-fold to 400-fold, 10-fold to 300-fold, or any range bounded by any of the values listed above. These increases are determined relative to the L-homocysteine titer observed in a L-homocysteine-producing microbial cell that does not include genetic alterations to reduce feedback regulation. This reference cell may (but need not) have other genetic alterations aimed at increasing L-homocysteine production, i.e., the cell may have increased activity of an upstream pathway enzyme.

In various embodiments, the L-homocysteine titers achieved by reducing feedback deregulation are at least 10, 20, 30, 40, 50, 75, 100, 200, 300, 400, 500, 600, 700, 800, or 900 μg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 mg/L. In various embodiments, the titer is in the range of 50 μg/L to 100 mg/L, 75 μg/L to 75 mg/L, 100 μg/L to 50 mg/L, 200 μg/L to 40 gm/L, 300 μg/L to 30 gm/L, 500 μg/L to 25 mg/L, 1 mg/L to 20 mg/L, or any range bounded by any of the values listed above.

Reduction of Consumption of L-Homocysteine and/or its Precursors

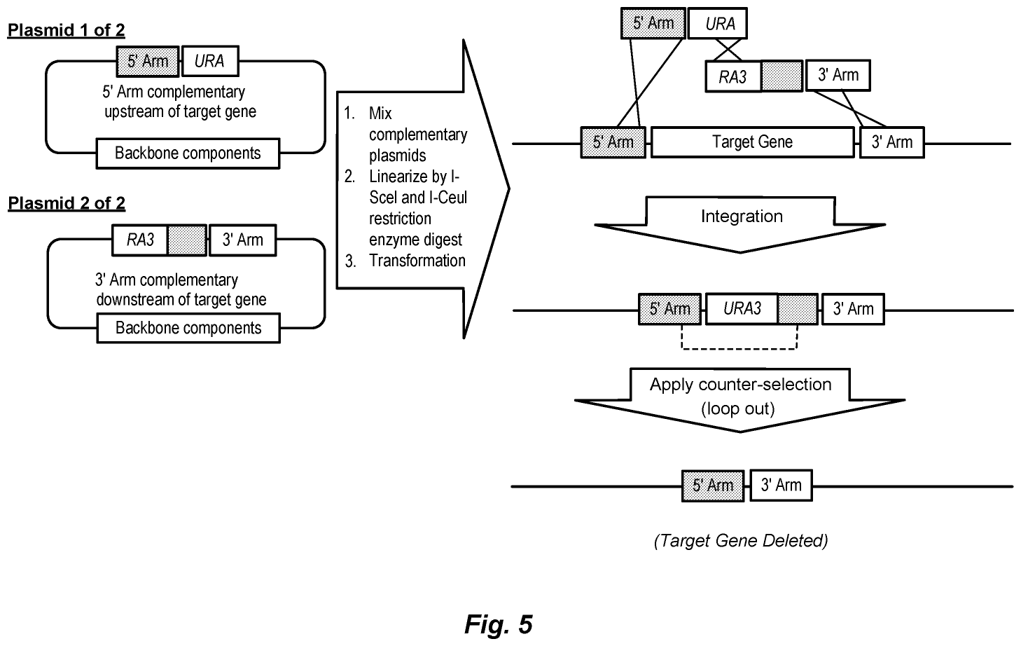

Another approach to increasing L-homocysteine production in a microbial cell that is capable of such production is to decrease the activity of one or more enzymes that consume one or more L-homocysteine pathway precursors or that consume L-homocysteine itself (see those discussed above in the Summary) In some embodiments, the activity of one or more such enzymes is reduced by modulating the expression or activity of the native enzyme(s). The activity of such enzymes can be decreased, for example, by substituting the native promoter of the corresponding gene(s) with a less active or inactive promoter or by deleting the corresponding gene(s). See FIGS. 4 and 5 for examples of schemes for promoter replacement and targeted gene deletion, respectively, in S. cervisiae and Y. lipolytica.

In various embodiments, the engineering of a L-homocysteine-producing microbial cell to reduce precursor consumption by one or more side pathways increases the L-homocysteine titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in L-homocysteine titer is in the range of 10-fold to 1000-fold, 20-fold to 500-fold, 50-fold to 400-fold, 10-fold to 300-fold, or any range bounded by any of the values listed above. These increases are determined relative to the L-homocysteine titer observed in a L-homocysteine-producing microbial cell that does not include genetic alterations to reduce precursor consumption. This reference cell may (but need not) have other genetic alterations aimed at increasing L-homocysteine production, i.e., the cell may have increased activity of an upstream pathway enzyme.

In various embodiments, the L-homocysteine titers achieved by reducing precursor consumption are at least 10, 20, 30, 40, 50, 75, 100, 200, 300, 400, 500, 600, 700, 800, or 900 μg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 mg/L. In various embodiments, the titer is in the range of 50 μg/L to 100 mg/L, 75 μg/L to 75 mg/L, 100 μg/L to 50 mg/L, 200 μg/L to 40 gm/L, 300 μg/L to 30 gm/L, 500 μg/L to 25 mg/L, 1 mg/L to 20 mg/L, or any range bounded by any of the values listed above.

Any of the approaches for increasing L-homocysteine production described above can be combined, in any combination, to achieve even higher L-homocysteine production levels.

Altering the Cofactor Specificity of Upstream Pathway Enzymes

Another approach to increasing L-homocysteine production in a microbial cell that is capable of such production is to alter the cofactor specificity of an upstream pathway enzyme that typically prefers the reduced form of nicotinamide adenine dinucleotide phosphate (NADPH) to the reduced from of nicotinamide adenine dinucleotide (NADH) (see those discussed above in the Summary). which provides the reducing equivalents for biosynthetic reactions. This can be achieved, for example, by expressing one or more variants of such enzymes that have the desired altered cofactor specificity. Examples of upstream pathway enzymes that rely on NADPH, and for which suitable variants are known, include aspartate semi-aldehyde dehydrogenase, homoserine dehydrogenase, and glyceraldehyde 3-phosphate dehydrogenase (GAPDH).

In various embodiments, the engineering of a L-homocysteine-producing microbial cell to alter the cofactor specificity of one or more of such enzymes increases the L-homocysteine titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in L-homocysteine titer is in the range of 10-fold to 1000-fold, 20-fold to 500-fold, 50-fold to 400-fold, 10-fold to 300-fold, or any range bounded by any of the values listed above. (Ranges herein include their endpoints.) These increases are determined relative to the L-homocysteine titer observed in a L-homocysteine-producing microbial cell that lacks any increase in activity of such enzymes. This reference cell may have one or more other genetic alterations aimed at increasing L-homocysteine production.

In various embodiments, the L-homocysteine titers achieved by altering the cofactor specificity of one or more enzymes that typically rely on NADPH as a cofactor are at least 10, 20, 30, 40, 50, 75, 100, 200, 300, 400, 500, 600, 700, 800, or 900 μg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 mg/L. In various embodiments, the titer is in the range of 50 μg/L to 100 mg/L, 75 μg/L to 75 mg/L, 100 μg/L to 50 mg/L, 200 μg/L to 40 gm/L, 300 μg/L to 30 gm/L, 500 μg/L to 25 mg/L, 1 mg/L to 20 mg/L, or any range bounded by any of the values listed above.

Illustrative Amino Acid and Nucleotide Sequences

The following table identifies amino acid and nucleotide sequences used in Examples 1 and 2. The corresponding sequences are shown in the Sequence Listing.

SEQ ID NO Cross-Reference Table

| | AA or DNA |
|---|---|
| Enzyme Description | SEQ ID NO: |
| Sulfate adenylyltransferase (ATP sulfurylase) from | 1 |
| Corynebacterium glutamicum (AA sequence) | |
| 5′-phosphosulfate sulfotransferase (PAPS reductase) | 2 |
| from Corynebacterium glutamicum (AA sequence) | |
| Sulfite reductase from Corynebacterium glutamicum | 3 |
| (AA sequence) | |
| Serine O-acetyltransferase from Lactobacillus acidophilus | 4 |
| (AA sequence) | |
| Feedback-Deregulated (G378E) Homoserine dehydrogenase | 5 |
| from Corynebacterium glutamicum (AA sequence) | |
| O-acetylhomoserine aminocarboxypropyltransferase from | 6 |
| Lactobacillus collinoides (AA sequence) | |
| Sulfate adenylyltransferase from Corynebacterium | 7 |
| glutamicum (AA sequence) | |
| Sulfate adenylyltransferase from Corynebacterium | 8 |
| glutamicum (DNA sequence encoding SEQ ID NO: 7) | |
| 5′-phosphosulfate sulfotransferase (PAPS reductase) from | 9 |
| Corynebacterium glutamicum (DNA sequence encoding | |
| SEQ ID NO: 2) | |
| Sulfite reductase from Corynebacterium glutamicum | 10 |
| (DNA sequence encoding SEQ ID NO: 3) | |
| Homoserine dehydrogenase from Corynebacterium | 11 |
| glutamicum (strain ATCC 13032/DSM 20300/JCM | |
| 1318/LMG 3730/NCIMB 10025); Uniprot ID: P08499 | |
| (AA sequence) | |

Microbial Host Cells

Any microbe that can be used to express introduced genes can be engineered for fermentative production of L-homocysteine as described above. In certain embodiments, the microbe is one that is naturally incapable of fermentative production of L-homocysteine. In some embodiments, the microbe is one that is readily cultured, such as, for example, a microbe known to be useful as a host cell in fermentative production of compounds of interest. Bacteria cells, including gram-positive or gram-negative bacteria can be engineered as described above. Examples include, in addition to C. glutamicum cells, Bacillus subtilus, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., P. alcaligenes, P. citrea, Lactobacilis spp. (such as L. lactis, L. plantarum), L. grayi, E. coli, E. faecium, E. gallinarum, E. casseliflavus, and/or E. faecalis cells.

There are numerous types of anaerobic cells that can be used as microbial host cells in the methods described herein. In some embodiments, the microbial cells are obligate anaerobic cells. Obligate anaerobes typically do not grow well, if at all, in conditions where oxygen is present. It is to be understood that a small amount of oxygen may be present, that is, there is some level of tolerance level that obligate anaerobes have for a low level of oxygen. Obligate anaerobes engineered as described above can be grown under substantially oxygen-free conditions, wherein the amount of oxygen present is not harmful to the growth, maintenance, and/or fermentation of the anaerobes.

Alternatively, the microbial host cells used in the methods described herein can be facultative anaerobic cells. Facultative anaerobes can generate cellular ATP by aerobic respiration (e.g., utilization of the TCA cycle) if oxygen is present. However, facultative anaerobes can also grow in the absence of oxygen. Facultative anaerobes engineered as described above can be grown under substantially oxygen-free conditions, wherein the amount of oxygen present is not harmful to the growth, maintenance, and/or fermentation of the anaerobes, or can be alternatively grown in the presence of greater amounts of oxygen.

In some embodiments, the microbial host cells used in the methods described herein are filamentous fungal cells. (See, e.g., Berka & Barnett, Biotechnology Advances, (1989), 7(2):127-154). Examples include Trichoderma longibrachiatum, T. viride, T. koningii, T. harzianum, Penicillium sp., Humicola insolens, H. lanuginose, H. grisea, Chrysosporium sp., C. lucknowense, Gliocladium sp., Aspergillus sp. (such as A. oryzae, A. niger, A. sojae, A. japonicus, A. nidulans, or A. awamori), Fusarium sp. (such as F. roseum, F. graminum F. cerealis, F. oxysporuim, or F. venenatum), Neurospora sp. (such as N. crassa or Hypocrea sp.), Mucor sp. (such as M. miehei), Rhizopus sp., and Emericella sp. cells. In particular embodiments, the fungal cell engineered as described above is A. nidulans, A. awamori, A. oryzae, A. aculeatus, A. niger, A. japonicus, T reesei, T. viride, F. oxysporum, or F. solani. Illustrative plasmids or plasmid components for use with such hosts include those described in U.S. Patent Pub. No. 2011/0045563.

Yeasts can also be used as the microbial host cell in the methods described herein. Examples include: Saccharomyces sp., Schizosaccharomyces sp., Pichia sp., Hansenula polymorpha, Pichia stipites, Kluyveromyces marxianus, Kluyveromyces spp., Yarrowia lipolytica and Candida sp. In some embodiments, the Saccharomyces sp. is S. cerevisiae (See, e.g., Romanos et al., Yeast, (1992), 8(6):423-488). Illustrative plasmids or plasmid components for use with such hosts include those described in U.S. Pat. No. 7,659,097 and U.S. Patent Pub. No. 2011/0045563.

In some embodiments, the host cell can be an algal cell derived, e.g., from a green alga, red alga, a glaucophyte, a chlorarachniophyte, a euglenid, a chromista, or a dinoflagellate. (See, e.g., Saunders & Warmbrodt, “Gene Expression in Algae and Fungi, Including Yeast,” (1993), National Agricultural Library, Beltsville, Md.). Illustrative plasmids or plasmid components for use in algal cells include those described in U.S. Patent Pub. No. 2011/0045563.

In other embodiments, the host cell is a cyanobacterium, such as cyanobacterium classified into any of the following groups based on morphology: Chlorococcales, Pleurocapsales, Oscillatoriales, Nostocales, Synechosystic or Stigonematales (See, e.g., Lindberg et al., Metab. Eng., (2010) 12(1):70-79). Illustrative plasmids or plasmid components for use in cyanobacterial cells include those described in U.S. Patent Pub. Nos. 2010/0297749 and 2009/0282545 and in Intl. Pat. Pub. No. WO 2011/034863.

Genetic Engineering Methods

Microbial cells can be engineered for fermentative L-homocysteine production using conventional techniques of molecular biology (including recombinant techniques), microbiology, cell biology, and biochemistry, which are within the skill of the art. Such techniques are explained fully in the literature, see e.g., “Molecular Cloning: A Laboratory Manual,” fourth edition (Sambrook et al., 2012); “Oligonucleotide Synthesis” (M. J. Gait, ed., 1984); “Culture of Animal Cells: A Manual of Basic Technique and Specialized Applications” (R. I. Freshney, ed., 6th Edition, 2010); “Methods in Enzymology” (Academic Press, Inc.); “Current Protocols in Molecular Biology” (F. M. Ausubel et al., eds., 1987, and periodic updates); “PCR: The Polymerase Chain Reaction,” (Mullis et al., eds., 1994); Singleton et al., Dictionary of Microbiology and Molecular Biology 2nd ed., J. Wiley & Sons (New York, N.Y. 1994).

Vectors are polynucleotide vehicles used to introduce genetic material into a cell. Vectors useful in the methods described herein can be linear or circular. Vectors can integrate into a target genome of a host cell or replicate independently in a host cell. For many applications, integrating vectors that produced stable transformants are preferred. Vectors can include, for example, an origin of replication, a multiple cloning site (MCS), and/or a selectable marker. An expression vector typically includes an expression cassette containing regulatory elements that facilitate expression of a polynucleotide sequence (often a coding sequence) in a particular host cell. Vectors include, but are not limited to, integrating vectors, prokaryotic plasmids, episomes, viral vectors, cosmids, and artificial chromosomes.

Illustrative regulatory elements that may be used in expression cassettes include promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods In Enzymology 185, Academic Press, San Diego, Calif. (1990).

In some embodiments, vectors may be used to introduce systems that can carry out genome editing, such as CRISPR systems. See U.S. Patent Pub. No. 2014/0068797, published 6 Mar. 2014; see also Jinek M., et al., “A programmable dual-RNA-guided DNA endonuclease in adaptive bacterial immunity,” Science 337:816-21, 2012). In Type II CRISPR-Cas9 systems, Cas9 is a site-directed endonuclease, namely an enzyme that is, or can be, directed to cleave a polynucleotide at a particular target sequence using two distinct endonuclease domains (HNH and RuvC/RNase H-like domains). Cas9 can be engineered to cleave DNA at any desired site because Cas9 is directed to its cleavage site by RNA. Cas9 is therefore also described as an “RNA-guided nuclease.” More specifically, Cas9 becomes associated with one or more RNA molecules, which guide Cas9 to a specific polynucleotide target based on hybridization of at least a portion of the RNA molecule(s) to a specific sequence in the target polynucleotide. Ran, F. A., et al., (“In vivo genome editing using Staphylococcus aureus Cas9,” Nature 520(7546):186-91, 2015, Apr. 9], including all extended data) present the crRNA/tracrRNA sequences and secondary structures of eight Type II CRISPR-Cas9 systems. Cas9-like synthetic proteins are also known in the art (see U.S. Published Patent Application No. 2014-0315985, published 23 Oct. 2014).

Example 1 describes illustrative integration approaches for introducing polynucleotides and other genetic alterations into the genomes of C. glutamicum cells.

Vectors or other polynucleotides can be introduced into microbial cells by any of a variety of standard methods, such as transformation, conjugation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. Transformants can be selected by any method known in the art. Suitable methods for selecting transformants are described in U.S. Patent Pub. Nos. 2009/0203102, 2010/0048964, and 2010/0003716, and International Publication Nos. WO 2009/076676, WO 2010/003007, and WO 2009/132220.

### Engineered Microbial Cells

The above-described methods can be used to produce engineered microbial cells that produce, and in certain embodiments, overproduce, L-homocysteine. Engineered microbial cells can have at least 1, 2, 3, 4, 5, 6, 7, 8, 9, 10, 20, 30, 40, 50, 60, 70, 80, 90, 100 or more genetic alterations, such as 30-100 alterations, as compared to a native microbial cell, such as any of the microbial host cells described herein. Engineered microbial cells described in the Example below have one, two, or three genetic alterations, but those of skill in the art can, following the guidance set forth herein, design microbial cells with additional alterations. In some embodiments, the engineered microbial cells have not more than 15, 14, 13, 12, 11, 10, 9, 8, 7, 6, 5, or 4 genetic alterations, as compared to a native microbial cell. In various embodiments, microbial cells engineered for L-homocysteine production can have a number of genetic alterations falling within the any of the following illustrative ranges: 1-10, 1-9, 1-8, 2-7, 2-6, 2-5, 2-4, 2-3, 3-7, 3-6, 3-5, 3-4, etc.

In some embodiments, an engineered microbial cell expresses at least one heterologous gene, e.g., a 3-phosphoadenosine-5-phosphosulfate sulfotransferase (PAPS reductase) gene, a sulfite reductase hemoprotein beta-component gene (sulfite reductase) gene, and/or or a sulfate adenylyltransferase subunit 1 gene (ATP sulfurylase) gene. In various embodiments, the microbial cell can include and express, for example: (1) a single heterologous PAPS reductase gene, (2) two or more heterologous PAPS reductase genes, which can be the same or different (in other words, multiple copies of the same heterologous PAPS reductase gene can be introduced or multiple, different heterologous PAPS reductase genes can be introduced), (3) a single heterologous PAPS reductase gene that is not native to the cell and one or more additional copies of an native PAPS reductase gene (if applicable), or (4) two or more non-native PAPS reductase genes, which can be the same or different, and one or more additional copies of a native PAPS reductase gene (if applicable). The same is true for other heterologous genes that can be introduced into the engineered microbial cell, such as those encoding sulfite reductase and/or ATP sulfurase.

This engineered host cell can include at least one additional genetic alteration that increases flux through any pathway leading to the production of an immediate precursor of L-homocysteine. As discussed above, this can be accomplished by one or more of the following: increasing the activity of upstream enzymes, reducing consumption of L-homocysteine precursors or of L-homocysteine itself, and altering the cofactor specificity of upstream pathway enzymes.

In addition, the engineered host cell can express an amino acid transporter to enhance transport of L-homocysteine from inside the engineered microbial cell to the culture medium.

The engineered microbial cells can contain introduced genes that have a native nucleotide sequence or that differ from native. For example, the native nucleotide sequence can be codon-optimized for expression in a particular host cell. Codon optimization for a particular host can, for example, be based on the codon usage tables found at www.kazusa.or.jp/codon/. The amino acid sequences encoded by any of these introduced genes can be native or can differ from native. In various embodiments, the amino acid sequences have at least 60 percent, 70 percent, 75 percent, 80 percent, 85 percent, 90 percent, 95 percent or 100 percent amino acid sequence identity with a native amino acid sequence.

The approach described herein has been carried out in bacterial cells, namely C. glutamicum. (See Example 1.)

Illustrative Engineered Bacterial Cells

In certain embodiments, the engineered bacterial (e.g., C. glutamicum) cell expresses one or more heterologous 3-phosphoadenosine-5-phosphosulfate sulfotransferases (PAPS reductases) having at least 70 percent, 75 percent, 80 percent, 85 percent, 90 percent, 95 percent or 100 percent amino acid sequence identity with an PAPS reductase encoded by a C. glutamicum 3-phosphoadenosine-5-phosphposulfate sulfotransferase gene; and/or one or more heterologous sulfite reductases having at least 70 percent, 75 percent, 80 percent, 85 percent, 90 percent, 95 percent or 100 percent amino acid sequence identity with a sulfite reductase encoded by a C. glutamicum sulfite reductase hemoprotein beta-component gene; and/or or one or more heterologous sulfate adenylyltransferases (ATP sulfurases) having at least 70 percent, 75 percent, 80 percent, 85 percent, 90 percent, 95 percent or 100 percent amino acid sequence identity with an ATP sulfurase encoded by a C. glutamicum sulfate adenylyltransferase subunit 1 gene or another C. glutamicum sulfate adenylyltransferase (e.g., SEQ ID NO:7).

In particular embodiments:

the PAPS reductase encoded by the C. glutamicum 3-phosphoadenosine-5-phosphposulfate sulfotransferase gene includes SEQ ID NO:2;

the sulfite reductase encoded by the C. glutamicum sulfite reductase hemoprotein beta-component gene includes SEQ ID NO:3; and/or

the ATP sulfurase encoded by the C. glutamicum sulfate adenylyltransferase subunit 1 gene includes SEQ ID NO:1.

In Corynebacteria glutamicum, for example, an about 18 mg/L titer of L-homocysteine was achieved by overexpressing the enzymes having SEQ ID NOs:1-3 (see Example 1).

In other particular embodiments:

the PAPS reductase encoded by the C. glutamicum 3-phosphoadenosine-5-phosphposulfate sulfotransferase gene includes SEQ ID NO:2;

the sulfite reductase encoded by the C. glutamicum sulfite reductase hemoprotein beta-component gene includes SEQ ID NO:3; and/or

the C. glutamicum sulfate adenylyltransferase includes SEQ ID NO:7.

In C. glutamicum, for example, an about 4.43 mg/L titer of L-homocysteine was achieved by overexpressing the enzymes having SEQ ID NOs:2, 3, and 7 (see Example 2).

Production in such strains can be increased by expressing additional genes encoding enzymes. In certain embodiments, the engineered bacterial (e.g., C. glutamicum) cell expresses, in addition to a set of the three enzymes described in the preceding paragraphs, one or more heterologous serine O-acetyltransferases having at least 70 percent, 75 percent, 80 percent, 85 percent, 90 percent, 95 percent or 100 percent amino acid sequence identity with a serine O-acetyltransferase from Lactobacillus acidophilus; and/or one or more heterologous homoserine dehydrogenases having at least 70 percent, 75 percent, 80 percent, 85 percent, 90 percent, 95 percent or 100 percent amino acid sequence identity with a homoserine dehydrogenase from C. glutamicum (e.g., strain ATCC 13032/DSM 20300/JCM 1318/LMG 3730/NCIMB 10025, Uniprot ID: P08499); and/or or one or more heterologous O-acetylhomoserine aminocarboxypropyltransferases having at least 70 percent, 75 percent, 80 percent, 85 percent, 90 percent, 95 percent or 100 percent amino acid sequence identity with an O-acetylhomoserine aminocarboxypropyltransferase from Lactobacillus collinoides.

In particular embodiments:

the serine O-acetyltransferase includes SEQ ID NO:4;

the homoserine dehydrogenase includes SEQ ID NO:11; and/or

the O-acetylhomoserine aminocarboxypropyltransferase includes SEQ ID NO:6.

In Corynebacteria glutamicum, for example, an about 73.7 mg/L titer of L-homocysteine was achieved by additionally expressing the enzymes having SEQ ID NOs:4, 11, and 6 (see Example 2).

### Culturing of Engineered Microbial Cells

Any of the microbial cells described herein can be cultured, e.g., for maintenance, growth, and/or L-homocysteine production.

In some embodiments, the cultures are grown to an optical density at 600 nm of 10-500, such as an optical density of 50-150.

In various embodiments, the cultures include produced L-homocysteine at titers of at least 10, 20, 30, 40, 50, 75, 100, 200, 300, 400, 500, 600, 700, 800, or 900 μg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 mg/L. In various embodiments, the titer is in the range of 50 μg/L to 100 mg/L, 75 μg/L to 75 mg/L, 100 μg/L to 50 gm/L, 200 μg/L to 25 gm/L, 300 μg/L to 10 gm/L, 350 μg/L to 5 gm/L or any range bounded by any of the values listed above.

Culture Media

Microbial cells can be cultured in any suitable medium including, but not limited to, a minimal medium, i.e., one containing the minimum nutrients possible for cell growth. Minimal medium typically contains: (1) a carbon source for microbial growth; (2) salts, which may depend on the particular microbial cell and growing conditions; and (3) water. Suitable media can also include any combination of the following: a nitrogen source for growth and product formation, a sulfur source for growth, a phosphate source for growth, metal salts for growth, vitamins for growth, and other cofactors for growth.

Any suitable carbon source can be used to cultivate the host cells. The term “carbon source” refers to one or more carbon-containing compounds capable of being metabolized by a microbial cell. In various embodiments, the carbon source is a carbohydrate (such as a monosaccharide, a disaccharide, an oligosaccharide, or a polysaccharide), or an invert sugar (e.g., enzymatically treated sucrose syrup). Illustrative monosaccharides include glucose (dextrose), fructose (levulose), and galactose; illustrative oligosaccharides include dextran or glucan, and illustrative polysaccharides include starch and cellulose. Suitable sugars include C6 sugars (e.g., fructose, mannose, galactose, or glucose) and C5 sugars (e.g., xylose or arabinose). Other, less expensive carbon sources include sugar cane juice, beet juice, sorghum juice, and the like, any of which may, but need not be, fully or partially deionized.

The salts in a culture medium generally provide essential elements, such as magnesium, nitrogen, phosphorus, and sulfur to allow the cells to synthesize proteins and nucleic acids.

Minimal medium can be supplemented with one or more selective agents, such as antibiotics.

To produce L-homocysteine, the culture medium can include, and/or is supplemented during culture with, glucose and/or a nitrogen source such as urea, an ammonium salt, ammonia, or any combination thereof.

Supplementation with a Reduced Sulfur Source

In particular embodiments, the medium can include a sulfur source that is in a reduced form, relative to sulfate, and/or the reduced sulfur source can be added to the culture during fermentation. In illustrative embodiments, the sulfur source includes a sulfide or thiosulfate. Examples of suitable reduced sulfur sources include sulfates, sulfites, sulfides, methylsulfonates, ametryne, prometryne, or any combinations thereof.

Culture Conditions

Materials and methods suitable for the maintenance and growth of microbial cells are well known in the art. See, for example, U.S. Pub. Nos. 2009/0203102, 2010/0003716, and 2010/0048964, and International Pub. Nos. WO 2004/033646, WO 2009/076676, WO 2009/132220, and WO 2010/003007, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass.

In general, cells are grown and maintained at an appropriate temperature, gas mixture, and pH (such as about 20° C. to about 37° C., about 6% to about 84% CO₂, and a pH between about 5 to about 9). In some aspects, cells are grown at 35° C. In certain embodiments, such as where thermophilic bacteria are used as the host cells, higher temperatures (e.g., 50° C.-75° C.) may be used. In some aspects, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the particular cell.

Standard culture conditions and modes of fermentation, such as batch, fed-batch, or continuous fermentation that can be used are described in U.S. Publ. Nos. 2009/0203102, 2010/0003716, and 2010/0048964, and International Pub. Nos. WO 2009/076676, WO 2009/132220, and WO 2010/003007. Batch and Fed-Batch fermentations are common and well known in the art, and examples can be found in Brock, Biotechnology: A Textbook of Industrial Microbiology, Second Edition (1989) Sinauer Associates, Inc.

In some embodiments, the cells are cultured under limited sugar (e.g., glucose) conditions. In various embodiments, the amount of sugar that is added is less than or about 105% (such as about 100%, 90%, 80%, 70%, 60%, 50%, 40%, 30%, 20%, or 10%) of the amount of sugar that can be consumed by the cells. In particular embodiments, the amount of sugar that is added to the culture medium is approximately the same as the amount of sugar that is consumed by the cells during a specific period of time. In some embodiments, the rate of cell growth is controlled by limiting the amount of added sugar such that the cells grow at the rate that can be supported by the amount of sugar in the cell medium. In some embodiments, sugar does not accumulate during the time the cells are cultured. In various embodiments, the cells are cultured under limited sugar conditions for times greater than or about 1, 2, 3, 5, 10, 15, 20, 25, 30, 35, 40, 50, 60, or 70 hours or even up to about 5-10 days. In various embodiments, the cells are cultured under limited sugar conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited sugar conditions can allow more favorable regulation of the cells.

In some aspects, the cells are grown in batch culture. The cells can also be grown in fed-batch culture or in continuous culture. Additionally, the cells can be cultured in minimal medium, including, but not limited to, any of the minimal media described above. The minimal medium can be further supplemented with 1.0% (w/v) glucose (or any other six-carbon sugar) or less. Specifically, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose. In some cultures, significantly higher levels of sugar (e.g., glucose) are used, e.g., at least 10% (w/v), 20% (w/v), 30% (w/v), 40% (w/v), 50% (w/v), 60% (w/v), 70% (w/v), or up to the solubility limit for the sugar in the medium. In some embodiments, the sugar levels falls within a range of any two of the above values, e.g.: 0.1-10% (w/v), 1.0-20% (w/v), 10-70% (w/v), 20-60% (w/v), or 30-50% (w/v). Furthermore, different sugar levels can be used for different phases of culturing. For fed-batch culture (e.g., of S. cerevisiae or C. glutamicum), the sugar level can be about 100-200 g/L (10-20% (w/v)) in the batch phase and then up to about 500-700 g/L (50-70% in the feed).

Additionally, the minimal medium can be supplemented 0.1% (w/v) or less yeast extract. Specifically, the minimal medium can be supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), or 0.02% (w/v) yeast extract. In some cultures, significantly higher levels of yeast extract can be used, e.g., at least 1.5% (w/v), 2.0% (w/v), 2.5% (w/v), or 3% (w/v). In some cultures (e.g., of S. cerevisiae or C. glutamicum), the yeast extract level falls within a range of any two of the above values, e.g.: 0.5-3.0% (w/v), 1.0-2.5% (w/v), or 1.5-2.0% (w/v).

Illustrative materials and methods suitable for the maintenance and growth of the engineered microbial cells described herein can be found below in Example 1.

### L-Homocysteine Production and Recovery

Any of the methods described herein may further include a step of recovering L-homocysteine. In some embodiments, the produced L-homocysteine contained in a so-called harvest stream is recovered/harvested from the production vessel. The harvest stream may include, for instance, cell-free or cell-containing aqueous solution coming from the production vessel, which contains L-homocysteine as a result of the conversion of production substrate by the resting cells in the production vessel. Cells still present in the harvest stream may be separated from the L-homocysteine by any operations known in the art, such as for instance filtration, centrifugation, decantation, membrane crossflow ultrafiltration or microfiltration, tangential flow ultrafiltration or microfiltration or dead-end filtration. After this cell separation operation, the harvest stream is essentially free of cells.

Further steps of separation and/or purification of the produced L-homocysteine from other components contained in the harvest stream, i.e., so-called downstream processing steps may optionally be carried out. These steps may include any means known to a skilled person, such as, for instance, concentration, extraction, crystallization, precipitation, adsorption, ion exchange, and/or chromatography. Any of these procedures can be used alone or in combination to purify L-homocysteine. Further purification steps can include one or more of, e.g., concentration, crystallization, precipitation, washing and drying, treatment with activated carbon, ion exchange, nanofiltration, and/or re-crystallization. The design of a suitable purification protocol may depend on the cells, the culture medium, the size of the culture, the production vessel, etc. and is within the level of skill in the art.

The following examples are given for the purpose of illustrating various embodiments of the disclosure and are not meant to limit the present disclosure in any fashion. Changes therein and other uses which are encompassed within the spirit of the disclosure, as defined by the scope of the claims, will be identifiable to those skilled in the art.

### Example 1—Construction and Selection of Strains of Corynebacteria glutamicum Engineered to Produce L-Homocysteine

Plasmid/DNA Design

All strains tested for this work were transformed with plasmid DNA designed using proprietary software. Plasmid designs were specific to each of the host organisms engineered in this work. The plasmid DNA was physically constructed by a standard DNA assembly method. This plasmid DNA was then used to integrate metabolic pathway inserts by one of two host-specific methods, each described below.

C. glutamicum and B. subtilis Pathway Integration

A “loop-in, single-crossover” genomic integration strategy has been developed to engineer C. glutamicum and B. subtilis strains. FIG. 10 illustrates genomic integration of loop-in only and loop-in/loop-out constructs and verification of correct integration via colony PCR. Loop-in only constructs (shown under the heading “Loop-in”) contained a single 2-kb homology arm (denoted as “integration locus”), a positive selection marker (denoted as “Marker”)), and gene(s) of interest (denoted as “promoter-gene-terminator”). A single crossover event integrated the plasmid into the C. glutamicum or B. subtilis chromosome. Integration events are stably maintained in the genome by growth in the presence of antibiotic (25 μg/ml kanamycin). Correct genomic integration in colonies derived from loop-in integration were confirmed by colony PCR with UF/IR and DR/IF PCR primers.

Loop-in, loop-out constructs (shown under the heading “Loop-in, loop-out) contained two 2-kb homology arms (5′ and 3′ arms), gene(s) of interest (arrows), a positive selection marker (denoted “Marker”), and a counter-selection marker. Similar to “loop-in” only constructs, a single crossover event integrated the plasmid into the chromosome. Note: only one of two possible integrations is shown here. Correct genomic integration was confirmed by colony PCR and counter-selection was applied so that the plasmid backbone and counter-selection marker could be excised. This results in one of two possibilities: reversion to wild-type (lower left box) or the desired pathway integration (lower right box). Again, correct genomic loop-out is confirmed by colony PCR. (Abbreviations: Primers: UF=upstream forward, DR=downstream reverse, IR=internal reverse, IF=internal forward.)

S. cerevisiae Pathway Integration

A “split-marker, double-crossover” genomic integration strategy has been developed to engineer S. cerevisiae strains. FIG. 7 illustrates genomic integration of complementary, split-marker plasmids and verification of correct genomic integration via colony PCR in S. cerevisiae. Two plasmids with complementary 5′ and 3′ homology arms and overlapping halves of a URA3 selectable marker (direct repeats shown by the hashed bars) were digested with meganucleases and transformed as linear fragments. A triple-crossover event integrated the desired heterologous genes into the targeted locus and re-constituted the full URA3 gene. Colonies derived from this integration event were assayed using two 3-primer reactions to confirm both the 5′ and 3′ junctions (UF/IF/wt-R and DR/IF/wt-F). For strains in which further engineering is desired, the strains can be plated on 5-FOA plates to select for the removal of URA3, leaving behind a small single copy of the original direct repeat. This genomic integration strategy can be used for gene knock-out, gene knock-in, and promoter titration in the same workflow.

Cell Culture

The workflow established for S. cerevisiae involved a hit-picking step that consolidated successfully built strains using an automated workflow that randomized strains across the plate. For each strain that was successfully built, up to four replicates were tested from distinct colonies to test colony-to-colony variation and other process variation. If fewer than four colonies were obtained, the existing colonies were replicated so that at least four wells were tested from each desired genotype.

The colonies were consolidated into 96-well plates with selective medium (SD-ura for S. cerevisiae) and cultivated for two days until saturation and then frozen with 16.6% glycerol at −80° C. for storage. The frozen glycerol stocks were then used to inoculate a seed stage in minimal media with a low level of amino acids to help with growth and recovery from freezing. The seed plates were grown at 30° C. for 1-2 days. The seed plates were then used to inoculate a main cultivation plate with minimal medium and grown for 48-88 hours. Plates were removed at the desired time points and tested for cell density (OD600), viability and glucose, supernatant samples stored for LC-MS analysis for product of interest.

Cell Density

Cell density was measured using a spectrophotometric assay detecting absorbance of each well at 600 nm. Robotics were used to transfer fixed amounts of culture from each cultivation plate into an assay plate, followed by mixing with 175 mM sodium phosphate (pH 7.0) to generate a 10-fold dilution. The assay plates were measured using a Tecan M1000 spectrophotometer and assay data uploaded to a LIMS database. A non-inoculated control was used to subtract background absorbance. Cell growth was monitored by inoculating multiple plates at each stage, and then sacrificing an entire plate at each time point.

To minimize settling of cells while handling large number of plates (which could result in a non-representative sample during measurement) each plate was shaken for 10-15 seconds before each read. Wide variations in cell density within a plate may also lead to absorbance measurements outside of the linear range of detection, resulting in underestimate of higher OD cultures. In general, the tested strains so far have not varied significantly enough for this be a concern.

Liquid-Solid Separation

To harvest extracellular samples for analysis by LC-MS, liquid and solid phases were separated via centrifugation. Cultivation plates were centrifuged at 2000 rpm for 4 minutes, and the supernatant was transferred to destination plates using robotics. 75 μL of supernatant was transferred to each plate, with one stored at 4° C., and the second stored at 80° C. for long-term storage.

Strategies for Enhancing Production of L-Homocysteine

Strategies for enhancing fermentative production of L-homocysteine in engineered microbes include one or more of the following:
Express feedback deregulated aspartate kinase (lysC).
Increase activity of aspartate transaminase.
Increase activity of glutamate dehydrogenase to provide glutamate for aspartate transaminase.
Increase activity of PEP carboxykinase; decrease activity of pyruvate carboxylase. This conserves ATP at the C3/C4 node which improves ATP availability for converting sulfate to sulfide.
Increase expression of sulfate transporter.
Increase activity of ATP sulfurylase.
Increase activity of homocysteine synthase.
Increase activity of homoserine dehydrogenase.
Increase activity of aspartate-semialdehyde dehydrogenase.
Increase activity of PAPS reductase.
Increase activity of sulfite reductase.
Decrease activity of cystathionine gamma-synthase. Conversion of O-acetyl-L-homoserine to L-homocysteine via L-cystathionine has lower yield compared to converting O-acetyl-L-homoserine to L-homocysteine via homocysteine synthase due to production of byproducts acetate, pyruvate and NH₃.
Decrease activity of cystathionine beta-synthase to decrease loss of L-homocysteine product.
Decrease activity of L-homoserine succinyl transferase.
Decrease activity of methionine synthase to decrease loss of L-homocysteine product.
Decrease activity of homoserine kinase.
Decrease activity of malate dehydrogenase.
Express a homoserine dehydrogenase having cofactor specificity switched from NADPH to NADH. This will decrease CO₂ loss due to generating NADPH thru the pentose phosphate pathway and improve yield.
Express an aspartate-semialdehyde dehydrogenase having cofactor specificity switched from NADPH to NADH. This will decrease CO₂ loss due to generating NADPH thru the pentose phosphate pathway and improve yield.
Express a PAPS reductase having cofactor specificity switched from NADPH to NADH. This will decrease CO₂ loss due to generating NADPH thru the pentose phosphate pathway and improve yield.
Express a sulfite reductase having cofactor specificity switched from NADPH to NADH. This will decrease CO₂ loss due to generating NADPH thru the pentose phosphate pathway and improve yield.
Utilize an alternative sulfur source (relative to sulfate) that is in a more reduced state, (e.g. sulfide, thiosulfate).

Results

C. glutamicum strain 7000139229 (CgHMCYS_12) was engineered to overproduce L-homocysteine. Strain 7000139229 by integration of Zymergen plasmid 13000234177 into the genome of publicly available C. glutamicum strain NRRL B-4263. Zymergen plasmid 13000234177 overexpresses the following: sulfate adenylyltransferase (ATP sulfurylase) subunit 1 gene from C. glutamicum (SEQ ID NO:1), 3-phosphoadenosine-5-phosphposulfate sulfotransferase (PAPS reductase) gene from C. glutamicum (SEQ ID NO: 2), and sulfite reductase hemoprotein beta-component gene from C. glutamicum (SEQ ID NO:3). Zymergen strain 7000139229 was cultivated in 96-well plates and showed production of 5-10 mg/L L-homocysteine.

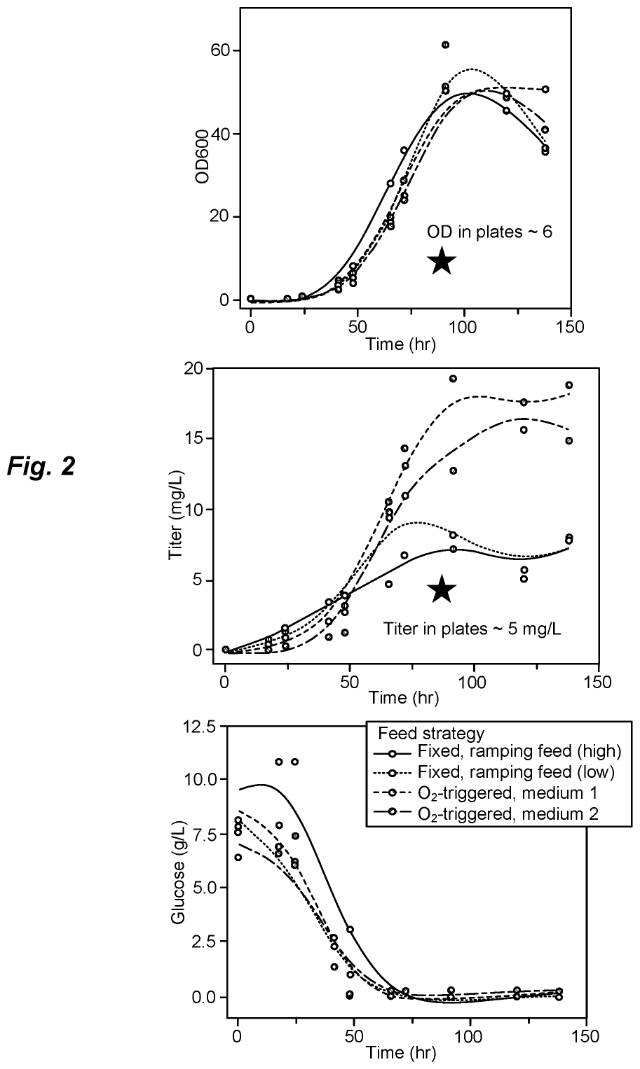

Subsequent cultivation in bioreactors, including process development, increased titers to ˜20 mg/L. Process development focused on fed-batch fermentation and a (minimal) medium composition that can support high cell densities and sugar inputs. FIG. 2 shows different fed-batch fermentation process configurations that started with a batch phase at 10 gm/L sugar, followed by a feed phase in which sugar (200 gm/L) is fed to the culture via different strategies. This approach achieved an increase cell density to OD₆₀₀ 50-60 and gave an L-homocysteine titer of ˜18 mg/L.

### Example 2—Further Engineering of Strains of Corynebacteria glutamicum to Produce L-Homocysteine

Two additional Corynebacteria glutamicum were designed, tested, and found to produce L-homocysteine.

CgHMCYS_56

CgHMCYS_56 was a C. glutamicum strain designed to express three enzymes: sulfate adenylyltransferase from C. glutamicum (SEQ ID NO:7), 5′-phosphosulfate sulfotransferase (PAPS reductase) from C. glutamicum (SEQ ID NO:2), sulfite reductase from C. glutamicum (SEQ ID NO: 3). This strain gave an L-homocysteine titer of 4.43 mg/L of culture medium.

CgHMCYS_127

CgHMCYS_127 was a C. glutamicum strain that expressed the same three enzymes as CgHMCYS_56 and additionally expressed the following enzymes: serine O-acetyltransferase from Lactobacillus acidophilus (Uniprot ID: A0A1D3PCK2) (SEQ ID NO:4), homoserine dehydrogenase from C. glutamicum (strain ATCC 13032/DSM 20300/JCM 1318/LMG 3730/NCIMB 10025) (Uniprot ID: P08499) (SEQ ID NO:11), and 0-acetylhomoserine aminocarboxypropyltransferase Lactobacillus collinoides, Uniprot ID: A0A166HL31 (SEQ ID NO:6). This strain produced L-homocysteine at a titer of 73.7 mg/L culture medium.

INFORMAL SEQUENCE LISTING
1> Sulfate adenylyltransferase (ATP) from Corynebacterium glutamicum
MTVPTLNKASEKIASRETLRLCTAGSVDDGKSTFVGRLLHDTKSVLADQLASVERTSADRGFEGLDLSLLVDGLRAEREQGI
TIDVAYRYFATDKRTFILADTPGHVQYTRNTVTGVSTSQVVVLLVDARHGVVEQTRRHLSVSALLGVRTVILAVNKIDLVDYS
EEVFRNIEKEFVSLASALDVTDTHVVPISALKGDNVAEPSTHMDWYAGPTVLEILENVEVSRGRAHDLGFRFPIQYVIREHAT
DYRGYAGTINAGSISVGDTVHLPEGRTTQVTHIDSADGSLQTASVGEAVVLRLAQEIDLIRGELIAGSDRPESVRSFNATVV
GLADRTIKPGAAVKVRYGTELVRGRVAAIERVLDIDGVNDNEAPETYGLNDIAHVRIDVAGELEVEDYAARGAIGSFLLIDQS
SGDTLAAGLVGHRLRNNWSI
2> 5′-phosphosulfate sulfotransferase (PAPS reductase) from Corynebacterium
glutamicum
MSFQLVNALKNTGSVKDPEISPEGPRTTTPLSPEVAKHNEELVEKHAAALYDASAQEILEWTAEHTPGAIAVTLSMENTVLA
ELAARHLPEADFLFLDTGYHFKETLEVARQVDERYSQKLVTALPILKRTEQDSIYGLNLYRSNPAACCRMRKVEPLAASLSP
YAGWITGLRRADGPTRAQAPALSLDATGRLKISPIITWSLEETNEFIADNNLIDHPLTHQGYPSIGCETCTLPVAEGQDPRAG
RWAGNAKTECGLHS
3> Sulfite reductase from Corynebacterium glutamicum
MTTTTGSARPARAARKPKPEGQWKIDGTEPLNHAEEIKQEEPAFAVKQRVIDIYSKQGFSSIAPDDIAPRFKWLGIYTQRKQ
DLGGELTGQLPDDELQDEYFMMRVRFDGGLASPERLRAVGEISRDYARSTADFTDRQNIQLHWIRIEDVPAIWEKLETVGL
STMLGCGDVPRVILGSPVSGVAAEELIDATPAIDAIRERYLDKEEFHNLPRKFKTAITGNQRQDVTHElQDVSFVPSIHPEFG
PGFECFVGGGLSTNPMLAQPLGSWIPLDEVPEVWAGVAGIFRDYGFRRLRNRARLKFLVAQWGIEKFREVLETEYLERKLI
DGPVVTTNPGYRDHIGIHPQKDGKFYLGVKPTVGHTTGEQLIAIADVAEKHGITRIRTTAEKELLFLDIERENLTTVARDLDEI
GLYSSPSEFRRGIISCTGLEFCKLAHATTKSRAIELVDELEERLGDLDVPIKIALNGCPNSCARTQVSDIGFKGQTVTDADGN
RVEGFQVHLGGSMNLDPNFGRKLKGHKVIADEVGEYVTRVVTHFKEQRHEDEHFRDWVQRAAEEDLV
4> Serine O-acetyltransferase from Lactobacillus acidophilus
MANKVKIGILNLMHDKLDTQSHFIKVLPNADLTFFYPRMHYQNRPIPPEVNMTSEPLDINRVSEFDGFIITGAPIDQIDFSKITYI
EEIRYLLQALDNHKIQQLYFCWGAMAALNYFYGIKKKILAEKIFGVFPHLITEPHPLLSGLSQGFMAPHARYAEMDKKQIMQD
ERLAINAVDDNSHLFMVSAKDNPERNFIFSHIEYGKDSLRDEYNREINAHPERHYKKPINYSMSNPSFQWQDTQKIFFNNW
LKKVKDNKLVLN
5> Feedback Deregulated (G378E) Homoserine dehydrogenase from Corynebacterium
glutamicum
MTSASAPSFNPGKGPGSAVGIALLGFGTVGTEVMRLMTEYGDELAHRIGGPLEVRGIAVSDISKPREGVAPELLTEDAFALI
EREDVDIVVEVIGGIEYPREVVLAALKAGKSVVTANKALVAAHSAELADAAEAANVDLYFEAAVAGAIPVVGPLRRSLAGDQI
QSVMGIVNGTTNFILDAMDSTGADYADSLAEATRLGYAEADPTADVEGHDAASKAAILASIAFHTRVTADDVYCEGISNISAA
DIEAAQQAGHTIKLLAICEKFTNKEGKSAISARVHPTLLPVSHPLASVNKSFNAIFVEAEAAGRLMFYGNGAGGAPTASAVLG
DVVGAARNKVHGGRAPGESTYANLPIADFGETTTRYHLDMDVEDRVEVLAELASLFSEQGISLRTIRQEERDDDARLIVVTH
SALESDLSRTVELLKAKPVVKAINSVIRLERD
6> O-acetylhomoserine aminocarboxypropyltransferase from Lactobacillus collinoides
MTDTSKLHFETKQVHAGQVVDETGARAVPIYQTTSYVFKDAAQAAGRFGLTDPGNIYTRLTNPTTDVLEKRVAQLENGTAG
VALATGAAAVTAAIENVANAGDNIVSASTLYGGTYDLFSVTLPKLGITTTFVDPDDPQNFAKAIDDKTKALYIETIGNPGINIIDI
EAVAKIAHDNGIVLIADNTFGTPYLIQPLDHGVDVVIHSATKFIGGHGTTMGGVIVENGKFDYAKSGKYPDFTTPDDTYNGIV
WNDINPATFTTKVRAQTLRDTGATISPFNSFLLLQGLESLSLRVERHVSNAQKIAKFLNDHPKVAWVNYPGLPG NKYNDLAK
KYFPKGTGSIFTIGLKGGEKAGKDLIEKLNLFSLLANVGDAKSLIIHPASTTHAQLNEEQLKETGITPDLIRLSIGIENVDDLIADL
SQALDQID
7> Sulfate adenylyltransferase from Corynebacterium glutamicum
MTTTVASELSPHLKDLENESIHILREVAGQFDKVGLLFSGGKDSVWYELARRAFAPANVPFELLHVDTGHNFPEVLEFRDN
LVERTGARLRVAKVQDWIDRGDLQERPDGTRNPLQTVPLVETIAEQGYDAVLGGARRDEERARAKERVFSVRDSFGGWD
PRRQRPELWTLYNGGHLPGENIRVFPISNWTEADIWEYIGARGIELPPIYFSHDREVFERDGMWLTAGEWGGPKKGEEIVT
KTVRYRTVGDMSCTGAVLSEARTIDDVIEEIATSTLTERGATRADDRLSESAMEDRKKEGYF
8> Sulfate adenylyltransferase from Corynebacterium glutamicum
atgaccacaaccgttgcatcagaactttccccacaccttaaagatcttgaaaatgaatccatccacatcctccgcgaggtagctggccagtttgata
aggtcggcctgctgttttccggcggtaaggattccgtcgtggtgtacgagcttgcgcgccgcgctttcgctccagctaacgtgccttttgaattgct
gcacgtggacaccggccacaacttcccagaggttttggaattccgcgacaacctggtggagcgcaccggcgcccgcctgcgcgtagctaaagtccag
gactggatcgatcgcggtgacctgcaggaacgcccagacggcacccgcaacccactgcagactgtccctttggtggagaccatcgctgagcagggct
acgacgccgtgcttggtggcgctcgccgcgatgaggagcgtgcccgcgccaaggagcgtgtgttctctgtgcgtgactccttcggtggttgggatcc
acgccgtcagcgcccagagctgtggaccctctacaacggtggccacctgccaggcgaaaacatccgtgttttcccaatctccaactggactgaagct
gacatctgggagtacatcggcgcccgtggcatcgaacttccaccgatctacttctcccacgaccgcgaagttttcgagcgcgacggcatgtggctga
ccgcaggcgagtggggtggaccaaagaagggcgaggagatcgtcaccaagactgttcgctaccgcaccgtcggcgatatgtcctgcaccggtgctgt
gctctcagaagcccgcaccattgacgatgtgatcgaagagatcgccacctccacccttaccgaacgtggcgcaacccgcgccgatgaccgcctcagc
gaatccgcaatggaagaccgcaagaaggaaggctacttc
9> 5'-phosphosulfate sulfotransferase (PAPS reductase) from Corynebacterium glutamicum
atgagctttcaactagttaacgccctgaaaaatactggttcggtaaaagatcccgagatctcacccgaaggacctcgcacgaccacaccgttgtcac
cagaggtagcaaaacacaacgaggaactcgtcgaaaagcatgctgctgcgttgtatgacgccagcgcgcaagagatcctggaatggacagccgagca
cacgccgggcgctattgcagtgaccttgagcatggaaaacaccgtactggcggagctggctgcgcggcacctgccggaagctgatttcctctifttg
gacaccggttaccacttcaaggaaactcttgaagttgcccgccaggtagatgagcgttattcccagaagcttgtcaccgcgctgccaatcctcaagc
gcacggagcaggattccatttatggtctcaacctgtaccgcagcaacccagcggcgtgctgccgaatgcgcaaagttgaaccgctggcggcgtcgtt
aagcccatacgctggctggatcaccggcctgcgccgcgctgatggcccaacccgtgctcaagcccctgcgctgagcttggatgccaccggcaggctc
aagatttctccaattatcacctggtcattggaggaaaccaacgagttcattgcggacaacaacctcatcgatcacccacttacccatcagggttatc
catcaattggatgcgaaacctgcacccttcctgttgctgaaggacaagaccctagggccggccgttgggctggaaacgccaagacagaatgcggact
tcactca
10> Sulfite reductase from Corynebacterium glutamicum
atgacaacaaccaccggaagtgcccggccagcacgtgccgccaggaagcctaagcccgaaggccaatggaaaatcgacggcaccgagccgcttaacc
atgccgaggaaattaagcaagaagaacccgcttttgctgtcaagcagcgggtcattgatatttactccaagcagggtttttcttccattgcaccgga
tgacattgccccacgctttaagtggttgggcatttacacccagcgtaagcaggatctgggcggtgaactgaccggtcagcttcctgatgatgagctg
caggatgagtacttcatgatgcgtgtgcgttttgatggcggactggcttcccctgagcgcctgcgtgccgtgggtgaaatttctagggattatgctc
gttccaccgcggacttcaccgaccgccagaacattcagctgcactggattcgtattgaagatgtgcctgcgatctgggagaagctagaaaccgtcgg
actgtccaccatgcttggttgcggtgacgttccacgtgttatcttgggctccccagtttctggcgtagctgctgaagagctgatcgatgccaccccg
gctatcgatgcgattcgtgagcgctacctagacaaggaagagttccacaaccttcctcgtaagtttaagactgctatcactggcaaccagcgccagg
atgttacccacgaaatccaggacgtttccttcgttccttcgattcacccagaattcggcccaggatttgagtgctttgtgggcggcggcctgtccac
caacccaatgcttgctcagccacttggttcttggattccacttgatgaggttccagaagtgtgggctggcgtcgccggaattttccgcgactacggc
ttccgacgcctgcgtaaccgtgctcgcctcaagttcttggtggcacagtggggtattgagaagttccgtgaagttcttgagaccgaatacctcgagc
gcaagctgattgatggcccagttgttaccaccaaccctggctaccgtgaccacattggcattcacccacaaaaggacggcaagttctacctcggtgt
gaagccaaccgttggacacaccaccggtgagcagctcattgccattgctgatgttgcagaaaagcacggcatcaccaggattcgtaccacggcggaa
aaggaactgctcttcctcgatattgagcgagagaaccttactaccgttgcacgtgacctggatgaaatcggactgtactcttcaccttccgagttcc
gccgcggcatcatttcctgcaccggcttggagttctgcaagcttgcgcacgcaaccaccaagtcacgagcaattgagcttgtggacgaactggaaga
gcgactcggcgatttggatgttcccatcaagattgccctgaacggttgccctaactcttgtgcacgcacccaggtttccgacatcggattcaaggga
cagaccgtcactgatgctgacggcaaccgcgttgaaggtttccaggttcacctgggcggttccatgaacttggatccaaacttcggacgcaagctca
agggccacaaggttattgccgatgaagtgggagagtacgtcactcgcgttgttacccacttcaaggaacagcgccacgaggacgagcacttccgcga
ttgggtccagcgggccgctgaggaagatttggtg
11> Homoserine dehydrogenase from Corynebacterium glutamicum
MTSASAPSFNPGKGPGSAVGIALLGFGTVGTEVMRLMTEYGDELANRIGGPLEVRGIAVSDISKPREGVAPELLTEDAFALI
EREDVDIVVEVIGGIEYPREVVLAALKAGKSVVTANKALVAAHSAELADAAEAANVDLYFEAAVAGAIPVVGPLRRSLAGDQI
QSVMGIVNGTTNFILDAMDSTGADYADSLAEATRLGYAEADPTADVEGHDAASKAAILASIAFHTRVTADDVYCEGISNISAA
DIEAAQQAGHTIKLLAICEKFTNKEGKSAISARVHPTLLPVSHPLASVNKSFNAIFVEAEAAGRLMFYGNGAGGAPTASAVLG
DVVGAARNKVHGGRAPGESTYANLPIADFGETTTRYHLDMDVEDRVEVLAELASLFSEQGISLRTIRQEERDDDARLIVVTH
SALESDLSRTVELLKAKPVVKAINSVIRLERD

What is claimed is:

1. An engineered microbial cell that comprises increased activity of at least one upstream pathway enzyme leading to L-homocysteine, wherein the at least one upstream pathway enzyme is selected from the group consisting of:
(a) 3-phosphoadenosine-5-phosphosulfate sulfotransferase (PAPS reductase),
(b) sulfite reductase, and
(c) sulfate adenylyltransferase (ATP sulfurase),
said increased activity being increased relative to a control cell, wherein the engineered microbial cell produces L-homocysteine.

2. The engineered microbial cell of claim 1, wherein the engineered microbial cell expresses at least two of said upstream pathway enzymes, wherein the at least two upstream pathway enzymes are selected from the group consisting of:
(a) a 3-phosphoadenosine-5-phosphosulfate sulfotransferase (PAPS reductase) and a sulfite reductase;
(b) a sulfite reductase and a sulfate adenylyltransferase (ATP sulfurase); and
(c) a 3-phosphoadenosine-5-phosphosulfate sulfotransferase (PAPS reductase) and a sulfate adenylyltransferase (ATP sulfurase).

3. The engineered microbial cell of claim 1 or claim 2, wherein said upstream pathway enzymes are heterologous enzymes.

4. The engineered microbial cell of claim 3, wherein the engineered microbial cell expresses:
(a) a heterologous 3-phosphoadenosine-5-phosphosulfate sulfotransferase (PAPS reductase);
(b) a heterologous sulfite reductase, and
(c) a heterologous sulfate adenylyltransferase (ATP sulfurase).

5. The engineered microbial cell of any one of claims 1-4, wherein the engineered microbial cell comprises increased activity of one or more additional upstream pathway enzyme(s) leading to L-homocysteine that is/are selected from the group consisting of phosphoadenosine phosphosulfate reductase (PAPS reductase), and homocysteine synthase, said increased activity being increased relative to a control cell.

6. The engineered microbial cell of any one of claims 1-5, wherein the engineered microbial cell comprises increased activity of a sulfate transporter, said increased activity being increased relative to a control cell.

7. The engineered microbial cell of any one of claims 1-6, wherein the engineered microbial cell comprises increased activity of one or more upstream pathway enzymes leading to O-acetyl-L-homoserine, said increased activity being increased relative to a control cell.

8. The engineered microbial cell of claim 7, wherein the one or more upstream pathway enzymes leading to O-acetyl-L-homoserine is/are selected from the group consisting of phosphoenolpyruvate carboxykinase (PEP carboxykinase), pyruvate kinase, pyruvate carboxylase, glutamate dehydrogenase, aspartate transaminase (aspartate aminotransferase), aspartate kinase (aspartokinase), aspartate-semialdehyde dehydrogenase, homoserine dehydrogenase, and L-homoserine-O-acetyltransferase.

9. The engineered microbial cell of claim 8, the one or more upstream pathway enzymes leading to O-acetyl-L-homoserine comprises PEP carboxykinase, and the activity of pyruvate carboxylase is reduced relative to a control cell.

10. The engineered microbial cell of any one of claims 1-9, wherein the activity of malate dehydrogenase is reduced relative to a control cell.

11. The engineered microbial cell of any one of claims 1-10, wherein the activity of the one or more upstream pathway enzymes is increased by expressing one or more feedback-deregulated enzyme(s).

12. The engineered microbial cell of claim 11, where the one or more feedback-deregulated enzyme (s) is/are selected from the group consisting of a feedback-deregulated aspartate kinase, a feedback-deregulated homoserine dehydrogenase, a feedback-deregulated aspartate-semialdehyde dehydrogenase, and a feedback-deregulated pyruvate carboxylase.

13. The engineered microbial cell of any one of claims 1-10, wherein the activity of the one or more upstream pathway enzymes is increased by expressing one or more upstream pathway enzyme(s) that is/are normally subject to feedback inhibition at the transcriptional level so as to reduce said feedback inhibition at the transcriptional level.

14. The engineered microbial cell of claim 13, wherein reduced feedback inhibition at the transcriptional level is achieved by a method comprising expressing aspartate kinase from a constitutive promoter.

15. The engineered microbial cell of any one of claims 1-14, wherein the engineered microbial cell comprises reduced activity of one or more enzyme(s) that consume one or more upstream pathway precursors, said reduced activity being reduced relative to a control cell.

16. The engineered microbial cell of claim 15, wherein the one or more enzyme(s) that consume one or more upstream pathway precursors is/are selected from the group consisting of cystathionine gamma-synthase, homoserine kinase, and L-homoserine succinyl transferase.

17. The engineered microbial cell of any one of claims 1-16, wherein the engineered microbial cell comprises reduced activity of one or more enzyme(s) that consume L-homocysteine, said reduced activity being reduced relative to a control cell.

18. The engineered microbial cell of claim 17, wherein the one or more enzyme(s) that consume L-homocysteine is/are selected from the group consisting of cystathionine beta-synthase and methionine synthase.

19. The engineered microbial cell of any one of claims 1-18, wherein the engineered microbial cell comprises reduced activity of one or more upstream pathway enzymes leading to cysteine, said reduced activity being reduced relative to a control cell.

20. The engineered microbial cell of claim 19, wherein the one or more upstream pathway enzymes leading to cysteine is/are selected from the group consisting of 3-phosphoglycerate dehydrogenase, phosphoserine transaminase, phosphoserine phosphatase, serine-O-acetyltransferase, and cysteine synthase.

21. The engineered microbial cell of any of claims 1-20, wherein the engineered microbial cell comprises altered cofactor specificity of one or more upstream pathway enzyme(s) from the reduced form of nicotinamide adenine dinucleotide phosphate (NADPH) to the reduced from of nicotinamide adenine dinucleotide (NADH).

22. The engineered microbial cell of claim 21, wherein the one or more upstream pathway enzyme(s) whose cofactor specificity is altered is/are selected from the group consisting of aspartate semi-aldehyde dehydrogenase, PAPS reductase, and sulfite reductase.

23. The engineered microbial cell of any one of claims 1-22, wherein the engineered microbial cell is a Corynebacteria glutamicum cell.

24. The engineered microbial cell of claim 23, wherein the engineered microbial cell is a Corynebacteria glutamicum cell that expresses:
(a) a heterologous Corynebacteria glutamicum 3-phosphoadenosine-5-phosphposulfate sulfotransferase (PAPS reductase) comprising SEQ ID NO:2;
(b) a heterologous Corynebacteria glutamicum sulfite reductase hemoprotein beta-component comprising SEQ ID NO:3; and
(c) a heterologous Corynebacteria glutamicum sulfate adenylyltransferase subunit 1 comprising SEQ ID NO:1.

25. The engineered microbial cell of claim 23, wherein the engineered microbial cell is a Corynebacteria glutamicum cell that expresses:
(a) a heterologous Corynebacteria glutamicum 3-phosphoadenosine-5-phosphposulfate sulfotransferase (PAPS reductase) comprising SEQ ID NO:2;
(b) a heterologous Corynebacteria glutamicum sulfite reductase hemoprotein beta-component comprising SEQ ID NO:3; and
(c) a heterologous Corynebacteria glutamicum sulfate adenylyltransferase comprising SEQ ID NO:7.

26. The engineered microbial cell of claim 25, wherein engineered microbial cell additionally expresses:
(a) a heterologous Lactobacillus acidophilus serine O-acetyltransferase comprising SEQ ID NO:4;
(b) a heterologous Corynebacteria glutamicum homoserine dehydrogenase comprising SEQ ID NO:11; and
(c) a heterologous Lactobacillus collinoides O-acetylhomoserine aminocarboxypropyltransferase comprising SEQ ID NO:6.

27. A culture of engineered microbial cells according to any one of claims 1-26, optionally wherein the culture comprises L-homocysteine at a level of at least 15 mg/L of culture medium.

28. A method of culturing engineered microbial cells according to any one of claims 1-26, the method comprising culturing the cells under conditions suitable for producing L-homocysteine, optionally wherein the method additionally comprises recovering L-homocysteine from the culture.
